SEQUENCE LISTING (9 sequences)

SEQ ID NO: 1 - protein, 774 aa, synthetic construct
MKSSHHHHHHGSSMKIEEGKLVIWINGDKGYNGLAEVGKKFEKDTGIKVTVEHPDKLEEKFPQVAATGDGPDIIFWAHDRFGGYAQSGLLAEITPDKAFQDKLYPFTWDAVRYNGKLIAYPIAVEALSLIYNKDLLPNPPKTWEEIPALDKELKAKGKSALMFNLQEPYFTWPLIAADGGYAFKYENGKYDIKDVGVDNAGAKAGLTFLVDLIKNKHMNADTDYSIAEAAFNKGETAMTINGPWAWSNIDTSKVNYGVTVLPTFKGQPSKPFVGVLSAGINAASPNKELAKEFLENYLLTDEGLEAVNKDKPLGAVALKSYEEELAKDPRIAATMENAQKGEIMPNIPQMSAFWYAVRTAVINAASGRQTVDEALKDAQTNSSSNNNNNNNNNNLGIEENLYFQSNAGGSSETGPVAVDPTLRRRIEPHEFEVFFDPRELRKETCLLYEINWGGRHSIWRHTSQNTNKHVEVNFIEKFTTERYFCPNTRCSITWFLSWSPCGECSRAITEFLSRYPHVTLFIYIARLYHHADPRNRQGLRDLISSGVTIQIMTEQESGYCWRNFVNYSPSNEAHWPRYPHLWVRLYVLELYCIILGLPPCLNILRRKQPQLTFFTIALQSCHYQRLPPHILWATGLKGSGTSGAGSATAGSGAGGQVQLVESGGGLVQAGDSLRLSCVASGRSLDGATMRWYRQAPGKEREFVAGIFWDEIGTEYADTAKGRFTISRDNAKNTIYLQMTNLRSEDTAMYYCNGLVFGGEYWGQGTQVTVSSGGC

SEQ ID NO: 2-3 - skipped (2 entries)

SEQ ID NO: 4 - protein, 18 aa, synthetic construct
GSGTSGAGSATAGSGAGG

SEQ ID NO: 5 - DNA, 15 bp, synthetic construct
  source 1..15: mol_type = other DNA
aaatatcatgcgcat

SEQ ID NO: 6 - DNA, 14 bp, synthetic construct
  source 1..14: mol_type = other DNA
tcaaatatcatgcg

SEQ ID NO: 7 - DNA, 35 bp, synthetic construct
  source 1..35: mol_type = other DNA
aaactagtgcatgaatagattttttcctgattatt

SEQ ID NO: 8 - DNA, 35 bp, synthetic construct
  source 1..35: mol_type = other DNA
aaactagtgcatgaatagattctctcctgattatt

SEQ ID NO: 9 - DNA, 48 bp, synthetic construct
  source 1..48: mol_type = other DNA
actgtaaagaatttagctgtatcaaactagtgcatgaatagattctct

(19) United States
(12) Patent Application Publication
(10) Pub. No.: US 2026/0308279 A1
(43) Pub. Date: Oct. 8, 2026

(54) METHODS AND COMPOSITIONS FOR IN SITU IDENTIFICATION OF PROTEIN-NUCLEIC ACID INTERACTION SITES

(71) Applicant: The Regents of the University of California, Oakland, CA (US)

(72) Inventors: Kevin D. Corbett, San Diego, CA (US); Eugene Yeo, La Jolla, CA (US); Qishan Liang, La Jolla, CA (US); Tao Yu, La Jolla, CA (US)

(21) Appl. No.: 19/141,785

(22) PCT Filed: Dec. 20, 2023
(86) PCT No.: PCT/US2023/085060

§ 371 Date: Jun. 20, 2025

Related U.S. Application Data
(60) Provisional application No. 63/434,886, filed on Dec. 22, 2022.

(51) Int. Cl. C07K 16/42; C12Q 1/6804; C12Q 1/6869

(52) U.S. Cl. CPC C07K 16/42; C12Q 1/6804; C12Q 1/6869; C07K 2317/22; C07K 2317/569; C07K 2319/61; C07K 2319/70

(57) ABSTRACT

Provided are methods and compositions the identification of binding sites on nucleic acids in situ by particular proteins or protein complexes. The methods and compositions allow binding site mapping without the need for specific labeling, tagging, or engineering of the proteins or cells being assessed.

### CROSS-REFERENCE TO RELATED APPLICATION

This application claims the benefit under 35 U.S.C. § 119(e) of U.S. Provisional Patent Application No. 63/434,886, filed Dec. 22, 2022, the entire contents of which are incorporated herein by reference in their entireties.

### STATEMENT OF GOVERNMENT SUPPORT

This invention was made with government support under HG011864 awarded by the National Institutes of Health. The government has certain rights in the invention.

### BACKGROUND

In existing methods for mapping protein-nucleic acid binding sites, the nucleic acids are sheared or cleaved, limiting the read length of subsequent sequencing steps. This limits the application of mapping technologies. This disclosure addresses this need and provides related advantages for diagnostic and therapeutic use of nucleic acids.

### SUMMARY OF THE DISCLOSURE

In one aspect, the present disclosure provides an engineered, non-naturally occurring recognition-editing system comprising a fusion polypeptide comprising: (a) a protein-specific recognition module, wherein the module specifically recognizes a target protein; and (b) a nucleic acid base editor domain, wherein the nucleic acid base editor domain is capable of (i) altering one or more specific bases of a nucleic acid to a different base; or (ii) modifying a specific nucleoside of a nucleic acid in a manner that is detectable by nucleic acid sequencing. In some embodiments, the engineered, non-naturally occurring recognition-editing system further comprises a supplementary recognition component. In some embodiments, the supplementary recognition component is a primary antibody that specifically binds to the target protein. In some embodiments the protein-specific recognition module is an antibody or an antigen binding fragment thereof, or a nanobody or an antigen binding fragment thereof, wherein the protein-specific recognition module specifically binds to the target protein. In some embodiments, the protein-specific recognition module is an antibody or an antigen binding fragment thereof, or a nanobody or an antigen binding fragment thereof, wherein the protein-specific recognition module specifically binds to the supplementary recognition component. In some embodiments, the protein-specific recognition module is a small molecule. In some embodiments, the protein-specific recognition module is capable of recognizing a tag fused to the target protein. In some embodiments, the nucleic acid base editor domain comprises an APOBEC family base editor or an ADAR family base editor. In some embodiments, the nucleic acid base editor domain comprises an APOBEC1 enzyme. In some embodiments, the protein-specific recognition module is a nanobody. In some embodiments, the nanobody specifically binds to Immunoglobulin G (IgG), e.g., human, mouse, or rabbit IgG. In some embodiments, the target protein is an RNA-binding protein (RBP). In some embodiments, the target protein is RBFOX2 or TAR DNA-binding protein 43 (TDP-43). In some embodiments, the fusion polypeptide comprises an amino acid sequence that is at least 70%, at least 75%, at least 80%, at least 85%, at least 90%, at least 95%, at least 99%, or 100% identical to SEQ ID NO: 1. Polynucleotides encoding the fusion polypeptide, vectors and host cells comprising the polynucleotides, and recombinant methods to prepare the fusion polypeptide are further disclosed herein.

In one aspect, the present disclosure provides a method of identifying one or more nucleic acid binding site(s) of a target protein or target protein complex in a biological sample, comprising, consisting essentially of, or yet further consisting of: (a) processing the biological sample, wherein processing comprises, consists essentially of, or yet consists of fixing and permeabilizing cells in the biological sample, wherein the biological sample is in one aspect a buffer; (b) contacting the biological sample with the engineered, non-naturally occurring recognition-editing system described herein, for a time sufficient to permit localization of the recognition-editing system to a target protein; (c) activating the nucleic acid base editor domain for a time sufficient to permit base editing of adjacent nucleic acids, thereby producing base edits; (d) extracting nucleic acids from the biological sample; (e) sequencing the nucleic acids in the biological samples, thereby generating first sequence data; and (f) profiling the base edits by identifying one or more sequence differences in the first sequence data relative to second sequence data obtained from a biological sample that was not contacted with the engineered, non-naturally occurring recognition-editing system, wherein the position of the one or more sequence differences represents one or more nucleic acid binding site of the target protein or the target protein complex. In some embodiments, the nucleic acid of the nucleic acid binding sites is DNA or RNA. In some embodiments, processing step further comprises, consists essentially of, or yet further consists of semi-permanently linking the target protein to its nucleic acid binding sites. In some embodiments, activating the nucleic acid base editor domain comprises, consists essentially of, or yet consists of, converting conditions of the buffer to promote enzymatic activity of the nucleic acid base editor domain. In some embodiments, sequencing comprises DNA sequencing or RNA sequencing. In some embodiments, sequencing comprises Sanger sequencing, short-read sequencing, long-read sequencing, or spatial transcriptomics. In some embodiments, contacting the biological sample with the engineered, non-naturally occurring recognition-editing system comprises: (i) contacting the biological sample with a supplementary recognition component for a time sufficient to permit the binding of the supplementary recognition component to the target protein; and (ii) contacting the biological sample with the fusion polypeptide for a time sufficient to permit the binding of the protein-specific recognition module to the supplementary recognition component. In some embodiments, contacting the biological sample with a supplementary recognition component is performed for about 1 hour. In some embodiments, contacting the biological sample with the fusion polypeptide is performed for about 1 hour. In some embodiments, activating the nucleic acid base editor domain is performed for about 16 hours.

In one aspect, the present disclosure provides a fusion polypeptide comprising, consisting essentially of, or yet consists of: (a) a protein-specific recognition module; and (b) a nucleic acid base editor domain, wherein the nucleic acid base editor domain is capable of (i) altering one or more specific bases of a nucleic acid to a different base; or (ii) modifying a specific nucleoside of a nucleic acid in a manner that is detectable by nucleic acid sequencing. In some embodiments, the protein-specific recognition module is an antibody or an antigen binding fragment thereof, or a nanobody or an antigen binding fragment thereof, wherein the protein-specific recognition module specifically binds to a target protein. In some embodiments, the protein-specific recognition module is an antibody or an antigen binding fragment thereof, or a nanobody or an antigen binding fragment thereof, wherein the protein-specific recognition module specifically binds to an Immunoglobulin G (IgG). In some embodiments, the protein-specific recognition module is a nanobody. In some embodiments, the nanobody specifically binds to an IgG, e.g., a rabbit, mouse, or human Immunoglobulin G (IgG). In some embodiments, the nucleic acid base editor domain comprises, consists essentially of, or yet consists of, an APOBEC family base editor or an ADAR family base editor. In some embodiments, the nucleic acid base editor domain comprises, consists essentially of, or yet consists of, an APOBEC1 enzyme. In some embodiments, the fusion polypeptide comprises, consists essentially of, or yet consists of, an amino acid sequence that is at least 70%, at least 75%, at least 80%, at least 85%, at least 90%, at least 95%, at least 99%, or 100% identical to SEQ ID NO: 1.

In one aspect, the present disclosure provides a polynucleotide encoding a fusion polypeptide of the disclosure, optionally further comprising one or more regulatory sequences operably linked to the polynucleotide.

In one aspect, the present disclosure provides a vector comprising a polynucleotide of the disclosure. In some embodiments, the vector is an expression vector, e.g., a viral vector or a plasmid.

In one aspect, the present disclosure provides a cell comprising a recognition-editing system, a fusion polypeptide, a polynucleotide, or a vector of the present disclosure. In some embodiments, the cell is a mammalian cell, such as for example a human cell. In some embodiments, the cell is a HEK293T cell.

In one aspect, the present disclosure provides a kit comprising one or more of a recognition-editing system, a fusion polypeptide, a polynucleotide, a vector, and a cell of the present disclosure.

Both the foregoing summary and the following description of the drawings and detailed description are exemplary and explanatory. They are intended to provide further details of the disclosure, but are not to be construed as limiting. Other objects, advantages, and novel features will be readily apparent to those skilled in the art from the following detailed description of the disclosure.

It should be appreciated that all combinations of the foregoing concepts and additional concepts discussed in greater detail below are provided as being part of the inventive subject matter disclosed herein and may be employed in any combination to achieve the benefits described herein.

### BRIEF DESCRIPTION OF THE DRAWINGS

FIG. 1 is a schematic showing an exemplary design of an in situ STAMP assay. Figure discloses SEQ ID NOS 5, 6 and 5, respectively, in order of appearance.

FIGS. 2A-2D show experimental data showing the purification, expression, targeting, and RNA editing of an APOBEC1-nanobody fusion protein. FIG. 2A shows a Coomassie stained gel containing purified APOBEC1-nanobody fusion protein. FIG. 2B shows two chromatograms generated following a single editing assay. The Sanger sequencing data reveals a high level of C-to-U editing at multiple sites on the RNA. Figure discloses SEQ ID NOS 7 and 8, respectively, in order of appearance. FIG. 2C is a panel of immunofluorescence (IF) images showing the co-localization of a primary antibody targeting RBFOXII and the recombinant APOBEC1-nanobody fusion protein in fixed HEK293T cells.

FIG. 2D shows a graph plotting the density of the distance of the C-to-U edited peak center to the closest RBFOXII binding motif UGCAUG.

FIGS. 3A-3B illustrate in situ RNA C-to-U editing mediated by recombinant APOBEC1-Nanobody (APO1-Nb). FIG. 3A shows an experimental procedure for in situ STAMP. After cell or tissue fixation, the RBP-targeting antibody (Ab) and the APO1-Nb are incubated sequentially, followed by RNA editing in situ at 37° C. overnight. RNAs are harvested for RNA-seq library preparation. FIG. 3B shows a data analysis workflow for in situ STAMP. The RNA sequences were aligned to the transcriptome, followed by C-to-U edit quantification by SAILOR and edit clusters identification by FLARE. SAILOR is described in Deffit et al., Elife. 2017 Sep. 19; 6:e28625. doi: 10.7554/eLife.28625, and is incorporated herein by reference in its entirety.

FIGS. 4A-4I show experimental data showing the establishment and optimization of APO1-Nb mediated RNA editing in situ. FIG. 4A shows a graph showing the results of size exclusion chromatography at the final step of APO1-Nb purification. The arrow indicated the dimerized His-MBP-APO1-Nb. FIG. 4B shows an SDS-PAGE image of the dimerized His-MBP-APO1-Nb confirmed the purity of the protein. FIG. 4C shows additional Sanger sequencing results of the in vitro RNA editing assay. No obvious C-to-U editing was observed at high salt (75 mM or 300 mM NaCl) at 37° C. or low salt at 25° C. Figure discloses “ACTGTAAAGAATTTAGCTGTATCAAACTAGTGCATGAATAGATTCTCT” as SEQ ID NO: 9. FIG. 4D shows immunofluorescence (IF) images and quantified global intensities and nuclear fractions of anti-RBFOX2 Ab in HEK293T cells with different Ab dilution ratios. 1:200 dilution (highlighted) from 1 mg/mL stock was chosen for the highest global intensity. The dots indicate the average of global intensities or nuclear fractions of individual cells while the error bars indicate standard deviations, n>400. FIG. 4E shows IF images and quantified global intensities and nuclear fractions of APO1-Nb in HEK293T cells with fixed anti-RBFOX2 Ab concentration (1:200 dilution). The global intensity of APO1-Nb decreased at higher APO1-Nb dilution factors, whereas the nuclear fraction increased and saturated at around 1:1000 dilution (from 1 mg/mL stock). The 1:1000 dilution was chosen with both relatively high global intensity and nuclear fraction since RBFOX2 localized in the nucleus. The dots indicate the average of global intensities or nuclear fractions of individual cells while the error bars indicate standard deviations, n>400. FIG. 4F shows quantified colocalization between APO1-Nb and anti-RBFOX2 Ab at fixed anti-RBFOX2 Ab concentration. The colocalization was significantly higher (p=0.000484, Z score=4.17) at 1:1000 dilution than at 1:50 dilution of APO1-Nb from 1 mg/mL stock. Bar plot indicate the average of Pearson's correlation coefficient of individual field of images, the error bars indicate standard deviations (CI=0.8412-0.8996; 0.9691-0.9833, alpha=0.01), n=5. FIG. 4G shows de novo motif discovery of in situ STAMP replicates identified the canonical RBFOX2 motif UGCAUG as the top motif in all RBFOX2-STAMP replicates, but not the negative controls (enzyme-only and buffer-only). FIG. 4H shows the enrichment of UGCAUG motif at confident edit clusters center with different enzyme (APO1-Nb) dilutions. Decreasing enzyme concentration (larger dilution factor) from 1:50 to 1:100, 1:200, 1:500 and 1:1000 enhanced the enrichment of UGCAUG, whereas further decreased enzyme concentration to 1:2000 reduced the enrichment. FIG. 4I shows additional IGV tracks of example RBFOX2 target genes (PKM and OGT) with 1500 bp windows showing RBFOX2 eCLIP peaks, RBFOX2 in situ STAMP, and the enzyme-only control STAMP. The overlay displays the edit fraction quantified by SAILOR in 3 replicates of in situ STAMP, alongside with the confident edit clusters identified by FLARE.

FIGS. 5A-5H show experimental data demonstrating that in situ STAMP can identify authentic RBFOX2 binding sites through confident C-to-U edit clusters. FIG. 5A shows a Sanger sequencing result of the in vitro RNA editing assay validated the C-to-U editing enzymatic activity of the recombinant APO1-Nb. Figure discloses “ACTGTAAAGAATTTAGCTGTATCAAACTAGTGCATGAATAGATTCTCT” as SEQ ID NO: 9. FIG. 5B shows an Integrative genome viewer (IGV) tracks showing RBFOX2 eCLIP peaks (HEK293T) on the target gene APP expanding a 1500 bp window, compared with the read coverage, SAILOR-quantified edit fraction and FLARE-determined edit clusters of three replicates of RBFOX2-STAMP, along with the enzyme-only negative controls. The confident edit clusters derived from three replicates were also shown. FIG. 5C shows that de novo motif discovery of in situ STAMP replicates identified the canonical RBFOX2 motif UGCAUG as the top motif in all RBFOX2-STAMP replicates, but not the negative controls (enzyme-only and buffer-only). FIG. 5D shows a distribution of the distance between the closest UGCAUG to confident edit clusters for in situ RBFOX2 STAMP replicates and confident edit clusters, enzyme-only control replicates and confident edit clusters, and RBFOX2 STAMP in live cells. A peak at 0 nt distance indicated the enrichment of UGCAUG in proximity to the edit cluster center, demonstrating the RBFOX2-binding driven editing. The RBFOX2-STAMP confident edit clusters presented much stronger enrichment of adjacent UGCAUG motif than the individual replicates. FIG. 5E shows the actual fraction of UGCAUG-containing RBFOX2-STAMP edit clusters (dots) was significantly higher than that of the 20 permuted clusters (box plot). The box shows the quartiles while the whiskers extend to show the rest of the distribution; the median is represented by the center line; outliners are not shown. The enrichment was defined as the ratio of actual fraction to the mean of the permuted-derived fraction. Z-scores: RBFOX2 in situ STAMP, 49.93; Enzyme-only, 0.43; RBFOX2 in cell STAMP, 23.08. FIG. 5F shows the actual fraction of eCLIP-overlapping RBFOX2-STAMP edit clusters (diamonds) was significantly higher than that of the 20 permuted clusters (box plot) but not the enzyme-only control, demonstrating edit clusters specifically captured RBFOX2 targeted RNA sites. The enrichment was defined as the ratio of actual fraction to the mean of the permuted-derived fraction. Z-scores: RBFOX2 in situ STAMP, 18.84; Enzyme only, 0.39; RBFOX2 in cell STAMP, 5.45. FIG. 5G shows IGV tracks showing RBFOX2 eCLIP peak on the target gene APP, compared with RBFOX2 in situ STAMP and the enzyme-only control STAMP for both PacBio long-read direct cDNA, and short-read (next-generation sequencing) outputs. FIG. 5H shows de novo motif analysis of RBFOX2-STAMP and enzyme-only control PacBio long reads for edits with a confidence score >0.5.

FIGS. 6A-6C shows experimental data showing a comparison of in situ STAMP performance at various sequencing depths. FIG. 6A shows a comparison of number of C-to-U edit clusters identified by FLARE for in situ RBFOX2 STAMP in MeOH-fixed HEK293T cells with different sequencing depths. The edit cluster number was proportionally associated with sequencing depth. FIG. 6B shows the canonical RBFOX2 binding motif UGCAUG was identified as the top motif in de novo motif analysis of RBFOX2-STAMP confident edit clusters irrespective of sequencing depths. FIG. 6C shows IGV tracks of representative RBFOX2 target gene (APP) examining the effect of sequencing depth on FLARE confident edit clusters. The tracks include eCLIP peaks, FLARE confident edit clusters of RBFOX2 in situ STAMP, and the enzyme-only control STAMP.

FIGS. 7A-7E show a comparison of in situ STAMP performance at various sequencing depths. FIG. 7A shows the enrichment of UGCAUG motif at confident edit clusters center with different sequencing depths. Increasing sequencing depth from 20 to 40, 60, 80, 100 million (M) reads enhanced the enrichment of UGCAUG, although the improvement from 80M to 100M was marginal. FIG. 7B shows a comparison of the distribution of STAMP edit clusters with different sequencing depths. Both the total counts and the percentage of intronic edit clusters of in RBFOX2 STAMP increased upon deeper sequencing, indicating the intronic RBFOX2 binding sites were revealed more thoroughly at higher sequencing depths. FIG. 7C shows IGV tracks of representative RBFOX2 target gene (APP) examining the effect of sequencing depth on SAILOR-quantified edit fraction and C-to-U editing pattern. The tracks include eCLIP peaks, 3-replicate overlay of SAILOR-quantified edit fraction of RBFOX2 in situ STAMP and the enzyme-only control STAMP.

FIG. 7D shows the actual fraction of UGCAUG-containing RBFOX2-STAMP edit clusters (dots) compared with that of the 20 permuted clusters (box plot) with different sequencing depths. The box shows the quartiles while the whiskers extend to show the rest of the distribution; the median is represented by the center line; outliners are not shown. The actual fraction and the enrichment were both increased in RBFOX2-STAMP and remained similar in enzyme-only control with deeper sequencing. Z-scores: RBFOX2 STAMP 100M, 80M, 60M, 40M, 20M: 60.88, 43.00, 49.20, 29.63, 12.28. Enzyme only 100M, 80M, 60M, 40M, 20M: 1.32, −0.11, −0.05, −0.50, −0.56. FIG. 7E shows a comparison of eCLIP-overlapping fraction with different sequencing depths (box plot). Regardless of sequencing depth, the same in situ STAMP experiment exhibited similar overlapped fraction and enrichment. The box shows the quartiles while the whiskers extend to show the rest of the distribution; the median is represented by the center line; outliners are not shown. Z-scores: RBFOX2 STAMP 100M, 80M, 60M, 40M, 20M: 25.44, 25.95, 19.45, 19.12, 12.32. Enzyme only 100M, 80M, 60M, 40M, 20M: 2.49, 2.07, 0.97, 19.12, 0.73.

FIGS. 8A-8E show experimental data supporting the identification of authentic TAR DNA-binding protein 43 (TDP-43) binding sites through confident C-to-U edit clusters using in situ STAMP. FIG. 8A shows the result of a de novo motif analysis of TDP-43-STAMP confident edit clusters, where the canonical TDP-43 binding motif UGUGUG was identified as the top motif. FIG. 8B shows the actual fraction of UGUGUG-containing TDP-43-STAMP edit clusters (dots) compared with that of the 20 permuted clusters (box plot). The box shows the quartiles while the whiskers extend to show the rest of the distribution; the median is represented by the center line; outliners are not shown. Z-scores: TDP-43 STAMP, 22.22; Enzyme only, 0.33. FIG. 8C shows the distribution of the nearest UGUGUG distance to clusters of confident edits reveals a peak at Ont for in situ TDP-43-STAMP, indicating an abundance of UGUGUG sequences in close proximity to the center of the edit cluster, which demonstrates TDP43-binding driven C-to-U editing. FIG. 8D shows the actual fraction of eCLIP-overlapping TDP-43-STAMP edit clusters (diamonds) compared to that of the 20 permuted clusters (box plot). The box shows the quartiles while the whiskers extend to show the rest of the distribution; the median is represented by the center line; outliners are not shown. Z-scores: TDP-43 STAMP, 15.61; Enzyme only, −0.46. FIG. 8E shows IGV tracks of example RBFOX2 target genes (TARDBP, NUCKS1, XPO1, FUS) with 1500 bp windows showing RBFOX2 eCLIP peaks, RBFOX2 in situ STAMP and the enzyme-only control STAMP. The 3-replicate overlay of SAILOR-quantified edit fraction of in situ STAMP is shown along with the FLARE-determined confident edit clusters.

FIGS. 9A-9B show experimental data showing that in situ STAMP exhibited comparable performance in PFA-fixed cells. FIG. 9A shows an RNA quality examination of harvested RNA from PFA-fixed HEK293T cells and MeOH-fixed HEK293T cells by TapeStation post in situ RNA editing. Despite exhibiting a lower average size, the RNAs derived from PFA-fixed cells remain suitable for library preparation for Illumina short read sequencing. FIG. 9B shows IGV tracks of example TDP-43 target genes (TARDBP, NUCKS1) with 1500 bp windows showing eCLIP peaks, in situ STAMP in PFA-fixed cells and the enzyme-only control. The overlay displays the edit fraction quantified by SAILOR in 3 replicates of in situ STAMP, alongside with the confident edit clusters identified by FLARE.

FIGS. 10A-10E show experimental data showing that in situ STAMP exhibited comparable performance in PFA-fixed cells. FIG. 10A shows de novo motif analysis of confident edit clusters that revealed the canonical RBFOX2 motif UGCAUG (top motif) or TDP-43 motif UGUGUG (2^{nd} top motif), for RBFOX2-STAMP or TDP-43-STAMP in PFA-fixed HEK293T cells respectively. FIG. 10B shows a density plot of the distance between edit clusters to closest UGCAUG (for RBFOX2-STAMP, upper panel) or UGUGUG (for TDP-43 STAMP, lower panel). In situ STAMP in MeOH-fixed and PFA-fixed cells presented no obvious distinction in canonical motif enrichment at the STAMP confident edit cluster center. FIG. 10C shows the actual fraction of motif-containing in situ STAMP edit clusters (dots) compared with that of the 20 permuted clusters (box plot). The box shows the quartiles while the whiskers extend to show the rest of the distribution; the median is represented by the center line; outliners are not shown. Top: RBFOX2 STAMP has PFA-fixed HEK293T cells. Z-scores: RBFOX2 in situ STAMP (PFA-fixed), 29.27; Enzyme only (PFA-fixed), 0.54; RBFOX2 in cell STAMP: 23.08; Bottom: TDP-43 STAMP in PFA-fixed HEK293T cells. Z-scores: TDP-43 STAMP (PFA-fixed), 4.61; Enzyme only (PFA-fixed), −1.02. FIG. 10D shows the actual fraction of eCLIP-overlapping STAMP edit clusters (diamonds) was significantly higher than that of the 20 permuted clusters (box plot), indicating the edit clusters specifically captured the RBP-targeted RNA sites. Top: RBFOX2 STAMP in PFA-fixed cells. Z-scores: RBFOX2 in situ STAMP (PFA-fixed), 18.67; Enzyme only (PFA-fixed), 1.36; RBFOX2 in cell STAMP: 5.45; Bottom: TDP-43 STAMP in PFA-fixed cells. Z-scores: TDP-43 in situ STAMP (PFA-fixed), 7.10; Enzyme only (PFA-fixed), −0.62. FIG. 10E shows IGV tracks of example RBFOX2 target gene (PKM, OGT) with 1500 bp windows showing eCLIP peaks, in situ STAMP in PFA-fixed cells and the enzyme-only control. The overlay displays the edit fraction quantified by SAILOR in 3 replicates of in situ STAMP, alongside with the confident edit clusters identified by FLARE.

FIGS. 11A-11E show experimental data showing that in situ STAMP revealed RNA targets of RBFOX2 in mouse brain tissue. FIG. 11A shows that de novo motif discovery of mouse brain tissue in situ STAMP replicates and confident edit clusters identified the canonical RBFOX2 motif UGCAUG as the top motif in all RBFOX2-STAMP replicates, but not the negative controls (enzyme-only and buffer-only). FIG. 11B shows a density plot of the distribution of the closest UGCAUG distance to confident edit clusters for in situ STAMP in mouse brain tissue and enzyme-only control. A peak at 0 nt distance indicated the enrichment of UGCAUG in proximity to the edit cluster center, demonstrating the RBFOX2-binding driven editing at in situ STAMP, but not the enzyme-only control. FIG. 11C shows the actual fraction of UGCAUG-containing mouse RBFOX2-STAMP edit clusters (dots) was significantly higher than that of the 20 permuted clusters (box plot). The box shows the quartiles while the whiskers extend to show the rest of the distribution; the median is represented by the center line; outliners are not shown. The enrichment was defined as the ratio of actual fraction to the mean of the permuted-derived fraction. Z-scores: RBFOX2 STAMP (Mouse Brain), 26.46; Enzyme only (Mouse Brain), 2.22. FIG. 11D shows the actual fraction of mouse eCLIP-overlapping mouse RBFOX2-STAMP edit clusters (diamonds) was significantly higher than that of the 20 permuted clusters (box plot) but not the enzyme-only control, demonstrating edit clusters specifically captured mouse RBFOX2 targeted RNA sites. The eCLIP experiment was performed in mouse whole brain. Z-scores: RBFOX2 STAMP (Mouse Brain), 41.69; Enzyme only (Mouse Brain), 4.96. FIG. 11E shows IGV tracks of example mouse RBFOX2 target genes (Ywhag, Ppp3ca, Ppp3r1, K1c1) with 1500 bp windows showing mouse RBFOX2 eCLIP peaks, mouse brain RBFOX2 in situ STAMP and the enzyme-only control STAMP. The overlay displays the edit fraction quantified by SAILOR in 3 replicates of in situ STAMP, alongside with the confident edit clusters identified by FLARE.

### DETAILED DESCRIPTION

Embodiments according to the present disclosure will be described more fully hereinafter. Aspects of the disclosure may, however, be embodied in different forms and should not be construed as limited to the embodiments set forth herein. Rather, these embodiments are provided so that this disclosure will be thorough and complete, and will fully convey the scope of the invention to those skilled in the art. The terminology used in the description herein is for the purpose of describing particular embodiments only and is not intended to be limiting.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the present application and relevant art and should not be interpreted in an idealized or overly formal sense unless expressly so defined herein. Although not explicitly defined below, such terms should be interpreted according to their common meaning.

The terminology used in the description herein is for the purpose of describing particular embodiments only and is not intended to be limiting of the invention. All publications, patent applications, patents and other references mentioned herein are incorporated by reference in their entirety. In case of conflict, the present specification, including definitions, will control. Other aspects are set forth within the claims that follow.

The practice of the present technology will employ, unless otherwise indicated, conventional techniques of tissue culture, immunology, molecular biology, microbiology, chemical engineering, and cell biology, which are within the skill of the art.

Unless the context indicates otherwise, it is specifically intended that the various features described herein can be used in any combination. Moreover, the disclosure also contemplates that in some embodiments, any feature or combination of features set forth herein can be excluded or omitted. To illustrate, if the specification states that a complex comprises components A, B, and C (or A, B, and/or C), it is specifically intended that any of A, B or C, or a combination thereof, can be omitted and disclaimed singularly or in any combination.

Unless explicitly indicated otherwise, all specified embodiments, features, and terms intend to include both the recited embodiment, feature, or term and biological equivalents thereof.

All numerical designations, e.g., pH, temperature, time, concentration, and molecular weight, including ranges, are approximations that can be varied (+) or (−) by increments of 1.0 or 0.1, as appropriate, or alternatively by a variation of +/−15%, or alternatively 10%, or alternatively 5%, or alternatively 2%. It is to be understood, although not always explicitly stated, that all numerical designations are preceded by the term “about”.

### Definitions

As used in the description of the invention and the appended claims, the singular forms “a,” “an” and “the” are intended to include the plural forms as well, unless the context clearly indicates otherwise.

The terms “substantially” and “about” are used herein to describe and account for small variations. When used in conjunction with an event or circumstance, the terms can refer to instances in which the event or circumstance occurs precisely as well as instances in which the event or circumstance occurs to a close approximation. When used in conjunction with a numerical value, the terms can refer to a range of variation of less than or equal to ±10% of that numerical value, such as less than or equal to ±5%, less than or equal to ±4%, less than or equal to ±3%, less than or equal to ±2%, less than or equal to ±1%, less than or equal to ±0.5%, less than or equal to ±0.1%, or less than or equal to ±0.05%. When referring to a first numerical value as “substantially” or “about” the same as a second numerical value, the terms can refer to the first numerical value being within a range of variation of less than or equal to ±10% of the second numerical value, such as less than or equal to ±5%, less than or equal to ±4%, less than or equal to ±3%, less than or equal to ±2%, less than or equal to ±1%, less than or equal to ±0.5%, less than or equal to ±0.1%, or less than or equal to ±0.05%. The terms or “acceptable,” “effective,” or “sufficient” when used to describe the selection of any components, ranges, dose forms, etc. disclosed herein intend that said component, range, dose form, etc. is suitable for the disclosed purpose.

Additionally, amounts, ratios, and other numerical values are sometimes presented herein in a range format. It is to be understood that such range format is used for convenience and brevity and should be understood flexibly to include numerical values explicitly specified as limits of a range, but also to include all individual numerical values or sub-ranges encompassed within that range as if each numerical value and sub-range is explicitly specified. For example, a ratio in the range of about 1 to about 200 should be understood to include the explicitly recited limits of about 1 and about 200, but also to include individual ratios such as about 2, about 3, and about 4, and sub-ranges such as about 10 to about 50, about 20 to about 100, and so forth.

Also as used herein, “and/or” refers to and encompasses any and all possible combinations of one or more of the associated listed items, as well as the lack of combinations when interpreted in the alternative (“or”).

As used herein, the term “comprising” is intended to mean that the compositions and methods include the recited elements, but not excluding others. “Consisting essentially of” when used to define compositions and methods, shall mean excluding other elements of any essential significance to the composition or method. “Consisting of” shall mean excluding more than trace elements of other ingredients for claimed compositions and substantial method steps. Examples and implementations defined by each of these transition terms are within the scope of this disclosure. Accordingly, it is intended that the methods and compositions can include additional steps and components (comprising) or alternatively including steps and compositions of no significance (consisting essentially of) or alternatively, intending only the stated method steps or compositions (consisting of).

As used herein, “optional” or “optionally” means that the subsequently described event or circumstance can or cannot occur, and that the description includes instances where the event or circumstance occurs and instances where it does not.

As used herein, the terms “RNA-binding protein” and “RBP” refer to proteins that bind to double or single-stranded RNA in cells, and participate in forming ribonucleoprotein (RNP) complexes. And RBP can contain one or more structural motifs, such as but not limited to RNA recognition motif (RRM), dsRNA binding domain, and zinc finger domain.

As used herein, the term “protein-specific recognition module” refers to a functional protein domain or protein fragment that is capable of specifically recognizing a target protein. As used herein, the term “specifically recognizes a target protein” means specifically binding to a target protein, or specifically binding to an intermediate agent (e.g., a protein (e.g., an antibody or fragment thereof), small molecule, etc.) that specifically binds to the target protein. A protein-specific recognition module may be part of a fusion polypeptide, such as a fusion polypeptide comprising another functional domain, such as a base editor domain. A protein-specific recognition module may be a small molecule or other chemical reagent that specifically recognizes and/or binds to a protein of interest. A protein-specific recognition module may be a primary antibody or an antigen binding fragment thereof that recognizes a protein of interest. A protein-specific recognition module may be antibody or fragment thereof that recognizes and/or specifically binds to an intermediate protein bound to a protein of interest. For example, a protein-specific recognition module may be antibody or fragment thereof that binds to an antibody that is bound to a target protein of interest. A protein-specific recognition module may be an antibody or fragment thereof, or other protein that recognizes a specific tag fused to a protein of interest. A protein-specific recognition module may be a single-domain antibody or an antigen binding fragment thereof. A protein-specific recognition module may be a single chain variable fragment (scFv). A protein-specific recognition module may be a nanobody or an antigen binding fragment thereof.

As used herein, the term “base editor domain” or “nucleic acid base editor domain” refers to a functional protein or protein fragment that is capable of modifying a base of a nucleic acid (i.e., DNA or RNA). Non-limiting examples of modifications of a base include changing the base to a different base (e.g., a change of an adenine (A) to a guanine (G), a change of an Adenosine (A) to an Inosine (I), a change of a Cytosine (C) to a Uracil (U), and adding or removing function groups to or from a base (e.g., adding a methyl group, etc.). In some embodiments, a modification is detectable by nucleic acid sequencing (e.g., DNA sequencing or RNA sequencing). An example of a base editor domain is an Apolipoprotein B mRNA Editing Catalytic Polypeptide-like (APOBEC) family base editor. The APOBEC family is a family of evolutionarily conserved cytidine deaminases. An APOBEC family base editor is a base editor that utilizes an APOBEC enzyme. Non-limiting examples of members of the APOBEC protein family include APOBEC1 (NCBI Reference Sequence: NP_001291495.1, “C->U-editing enzyme APOBEC-1 isoform a [Homo sapiens],” APOBEC2 (NCBI Reference Sequence: NP_006780.1, “C->U-editing enzyme APOBEC-2 [Homo sapiens]”), APOBEC3A (NCBI Reference Sequence: NP_001257335.1, “DNA dC->dU-editing enzyme APOBEC-3A isoform b [Homo sapiens]”), APOBEC3B (NCBI Reference Sequence: NP_001257340.2, “DNA dC->dU-editing enzyme APOBEC-3B isoform b [Homo sapiens]”), APOBEC3C (NCBI Reference Sequence: NP_055323.2, “DNA dC->dU-editing enzyme APOBEC-3C [Homo sapiens]”), APOBEC3C (also known as APOBEC3E) (NCBI Reference Sequence: NP_689639.2, “DNA dC->dU-editing enzyme APOBEC-3D isoform 1 [Homo sapiens]”), APOBEC3F (NCBI Reference Sequence: NP_001006667.1, “DNA dC->dU-editing enzyme APOBEC-3F isoform b [Homo sapiens]”), APOBEC3G (NCBI Reference Sequence: NP_068594.1, “DNA dC->dU-editing enzyme APOBEC-3G isoform 1 [Homo sapiens]”), APOBEC3H (NCBI Reference Sequence: NP_001159474.2, “DNA dC->dU-editing enzyme APOBEC-3H isoform SV-182 [Homo sapiens]”), APOBEC4 (NCBI Reference Sequence: NP_982279.1, “putative C->U-editing enzyme APOBEC-4 [Homo sapiens]”), and Activation-induced (cytidine) deaminase (AID) (NCBI Reference Sequence: NP_001317272.1, “single-stranded DNA cytosine deaminase isoform 2 [Homo sapiens]”). An APOBEC enzyme of any species can be used in accordance with this disclosure. In some embodiments, a mammalian APOBEC enzyme, such as a rat APOBEC enzyme (e.g., a rat APOBEC1 enzyme) or a human enzyme is used (see e.g., https://www.uniprot.org/uniprotkb/P41238/entry). Another example of a base editor domain is an Adenosine Deaminase Acting on RNA (ADAR) family base editor. ADAR enzymes bind to double-stranded RNA (dsRNA) and convert adenosine to inosine (hypoxanthine) by deamination. An ADAR family base editor is a base editor that utilizes an ADAR enzyme. Non-limiting examples of members of the ADAR protein family include ADAR1 (NCBI Reference Sequence: NP_001020278.1, “double-stranded RNA-specific adenosine deaminase isoform d [Homo sapiens]”), ADARB1 (ADAR2) (NCBI Reference Sequence: NP_001103.1, “double-stranded RNA-specific editase 1 isoform 1 [Homo sapiens]”), and ADARB2 (ADAR3) (NCBI Reference Sequence: NP_061172.1, “double-stranded RNA-specific editase B2 [Homo sapiens]”).

As used herein, the term “RBFOX2” or “RBFOXII,” also known as RNA binding motif protein 9 (RBM9), refers to an RNA binding protein that is thought to be a key regulator of alternative splicing in the nervous system and other cell types. RBFOX2 and the related protein RBFOX1 bind to conserved (U)GCAUG RNA motifs in the introns adjacent to many alternatively spliced exons and promotes inclusion or exclusion of the alternative exon in mature RNA transcripts. An exemplary amino acid sequence of RBFOX2 is shown as NCBI Reference Sequence: NP_001026865.1, “RNA binding protein fox-1 homolog 2 isoform 1 [Homo sapiens].”

As used herein, the term “supplementary recognition component” refers to an agent that aids in the recognition of a target protein, but that is itself not physically linked to a base editor domain. For example, a supplementary recognition component can include a primary antibody or fragment thereof that specifically binds to the target protein. A primary antibody can be an antibody from any species. A primary antibody can be monoclonal or polyclonal. A primary antibody can be, for example, a mouse primary antibody, a rabbit primary antibody, a goat primary antibody, or a chicken primary antibody. In some embodiments, a primary antibody can be a humanized antibody. In a scenario in which a supplementary recognition component is used, the fusion protein of the recognition-editing system may include a nucleic acid base editor domain fused to a secondary antibody or fragment thereof that is capable of binding to the primary antibody or fragment thereof of the supplementary recognition component.

As used herein, the term “polynucleotide” refers to a polymeric form of nucleotides of any length, including deoxyribonucleotides or ribonucleotides, or analogs thereof. A polynucleotide may comprise modified nucleotides, such as methylated or capped nucleotides and nucleotide analogs, and may be interrupted by non-nucleotide components. If present, modifications to the nucleotide structure may be imparted before or after assembly of the polymer. The term polynucleotide, as used herein, refers interchangeably to double- and single-stranded molecules. Unless otherwise specified or required, any embodiment of the invention described herein that is a polynucleotide encompasses both the double-stranded form and each of two complementary single-stranded forms known or predicted to make up the double-stranded form.

The terms “polypeptide” and “protein” are used interchangeably herein to refer to polymers of amino acids of any length. The terms also encompass an amino acid polymer that has been modified; for example, disulfide bond formation, glycosylation, acetylation, phosphorylation, lipidation, or conjugation with a labeling component. Polypeptides, when discussed in the context of gene therapy and compositions therefor, refer to the respective intact polypeptide, or any fragment or genetically engineered derivative thereof that retains the desired biochemical function of the intact protein.

A “vector” as used herein refers to a macromolecule or association of macromolecules that comprises or associates with a polynucleotide and which can be used to mediate delivery of the polynucleotide to a cell, either in vitro or in vivo. Illustrative vectors include, for example, plasmids, viral vectors, liposomes, and other gene delivery vehicles. The polynucleotide to be delivered, sometimes referred to as a “target polynucleotide” or “transgene,” may comprise a coding sequence of interest in gene therapy (such as a gene encoding a protein of therapeutic or interest), a coding sequence of interest in vaccine development (such as a polynucleotide expressing a protein, polypeptide or peptide suitable for eliciting an immune response in a mammal), and/or a selectable or detectable marker.

An “expression vector” is a vector comprising a region which encodes a polypeptide of interest, and is used for effecting the expression of the protein in an intended target cell. An expression vector also comprises control elements operatively linked to the encoding region to facilitate expression of the protein in the target. The combination of control elements and a gene or genes to which they are operably linked for expression is sometimes referred to as an “expression cassette,” a large number of which are known and available in the art or can be readily constructed from components that are available in the art.

As used herein, the term “operably linked” refers to an arrangement of elements where the components so described are configured so as to perform their usual function. Thus, a given promoter operably linked to a coding sequence is capable of effecting the expression of the coding sequence when active. The promoter need not be contiguous with the coding sequence, so long as it functions to direct the expression thereof. Thus, for example, intervening untranslated yet transcribed sequences can be present between the promoter sequence and the coding sequence and the promoter sequence can still be considered “operably linked” to the coding sequence.

Carriers include pharmaceutical excipients and additives proteins, peptides, amino acids, lipids, and carbohydrates (e.g., sugars, including monosaccharides, di-, tri, tetra-oligosaccharides, and oligosaccharides; derivatized sugars such as alditols, aldonic acids, esterified sugars and the like; and polysaccharides or sugar polymers), which can be present singly or in combination, comprising alone or in combination 1-99.99% by weight or volume. Exemplary protein excipients include serum albumin such as human serum albumin (HSA), recombinant human albumin (rHA), gelatin, casein, and the like. Representative amino acid/antibody components, which can also function in a buffering capacity, include alanine, arginine, glycine, arginine, betaine, histidine, glutamic acid, aspartic acid, cysteine, lysine, leucine, isoleucine, valine, methionine, phenylalanine, aspartame, and the like. Carbohydrate excipients are also intended within the scope of this technology, examples of which include but are not limited to monosaccharides such as fructose, maltose, galactose, glucose, D-mannose, sorbose, and the like; disaccharides, such as lactose, sucrose, trehalose, cellobiose, and the like; polysaccharides, such as raffinose, melezitose, maltodextrins, dextrans, starches, and the like; and alditols, such as mannitol, xylitol, maltitol, lactitol, xylitol sorbitol (glucitol) and myoinositol.

As used herein, the term “contacting” means direct or indirect binding or interaction between two or more molecules. A particular example of direct interaction is binding. A particular example of an indirect interaction is where one entity acts upon an intermediary molecule, which in turn acts upon the second referenced entity. Contacting as used herein includes in solution, in solid phase, in vitro, ex vivo, in a cell and in vivo. Contacting in vivo can be referred to as administering, or administration.

### Modes For Carrying Out The Disclosure

RNA-binding proteins (RBPs) hold pivotal roles in RNA metabolism including splicing, stability, localization, and translation. Profiling RBP-RNA interactions across the transcriptome has promising diagnostic potential for diseases. However, current methods have limitations: immunoprecipitation (IP)-based technologies require substantial input materials, and are not isoform-sensitive, while RNA-modifying enzyme approaches require artificial fusion protein expression, possibly distorting interactions. The present disclosure addresses these short comings by providing an RBP-RNA profiling approach, in situ Surveying Targets by APOBEC-Mediated Profiling (STAMP), that employs, in some embodiments, a fusion of cytosine deaminase APOBEC1 and an IgG-targeting single-domain antibody (nanobody). This fusion protein can be precisely directed to RBPs of interest like RBFOX2 and TAR DNA-binding protein 43 (TDP-43) when combined with specific primary antibodies. This synergy enables site identification in unmodified HEK293T cells. High specificity is evident as in situ STAMP accurately identifies canonical binding motifs for RBFOX2 (UGCAUG) and TDP-43 (UGUGUG) through de novo motif analysis. The technique is compatible with cDNA PacBio long-read sequencing, potentially allowing the distinction between RNA isoforms while conserving RNA integrity. Crucially, in situ STAMP is adaptable to various fixation methods, such as methanol and formaldehyde, making it applicable to tissue samples. This breakthrough empowers the exploration of genuine RBP-RNA interactions using limited primary cells or tissues. This addresses a crucial gap in unveiling RBP roles in RNA-linked diseases within authentic biological contexts. Applicant describes herein the functional and/or physical linkage of a DNA or RNA base editor to a means for specific recognition of a protein or protein complex in situ. Base editors can include APOBEC family enzymes with RNA- or DNA-editing specificity, ADAR enzymes, or other engineered base editors. Specific protein recognition can be achieved in a number of ways, examples being (1) fusing the base editor directly to an antibody or nanobody that specifically recognizes the protein of interest; (2) fusing the base editor to an intermediate protein (nanobody, Protein A) that can specifically recognize an exogenously-added primary antibody against the protein of interest; (3) fusing the base editor to a small molecule that specifically binds the protein of interest.

This disclosure also enables the identification of a protein or protein complex's binding sites on nucleic acids (DNA or RNA) in situ (within cells or other biological milieu), without the need for specific labeling, tagging, or engineering of the proteins or cells in question. The method is applied to diverse samples including tissues or single cells from prokaryotes (bacteria or archaea) or eukaryotes (including mammals or non-mammal eukaryotes). In different formulations, the method enables identification of a protein or protein-RNA complex interaction sites on diverse nucleic acids including genomic DNA, extrachromosomal DNA, circular RNAs, messenger RNAs, long non-coding RNAs, tRNAs, viral DNA, or viral RNA. The method does not require engineering of the samples in question, enabling the examination of primary cells, patient samples, and wild-type organisms.

Applicant has discovered that one key innovation is the functional and/or physical linkage of a DNA or RNA base editor to a means for specific recognition of a protein or protein complex in situ. Base editors can include APOBEC family enzymes with RNA- or DNA-editing specificity, ADAR enzymes, or other engineered base editors. Specific protein recognition can be achieved in a number of ways, examples being (1) fusing the base editor directly to an antibody or nanobody that specifically recognizes the protein of interest; (2) fusing the base editor to an intermediate protein (nanobody, Protein A) that can specifically recognize an exogenously-added primary antibody against the protein of interest; (3) fusing the base editor to a small molecule that specifically binds the protein of interest.

Applicant provides herein methods wherein the nucleic acids are not cleaved, enabling long reads and in turn enabling, for example, new target-identification criteria including (for RNA) mRNA isoform identification, and (for DNA) haplotype-specific mapping (phasing). It provides in one aspect, more native conditions of the cells and is applicable to primary tissues. This also allows for a broader range of proteins that could be profiled as compared to the current state-of-the-art method, eCLIP, as this method does not require high grade, e.g., IP grade antibodies. In situ editing will also enable spatial information profiling when combined with spatial transcriptomic sequencing methods.

The disclosed method is a straightforward and flexible alternative to many current methods for mapping DNA or RNA binding sites for proteins of interest. The technology can be applied to the development of kits for the specific detection of medically-relevant proteins to DNA or RNA in patient samples, or more flexible kits for mapping any individual protein of interest on DNA or RNA to be marketed to researchers.

### Engineered Recognition-Editing Systems

Provided herein are engineered, non-naturally occurring recognition editing systems, and methods of using the same. The provided systems are useful for, inter alia, profiling protein-nucleic acid interactions, such as transcriptomic RBP-RNA interaction profiling.

This disclosure harnesses the combination of a protein-specific recognition module to a DNA/RNA base editor to label the DNA/RNA binding site of a protein of interest. The recognition module may be (1) a small molecule or other chemical reagent that specifically recognizes a protein of interest, (2) a primary antibody that recognizes a protein of interest, (3) an antibody that recognizes an intermediate protein (e.g., a primary antibody), that recognizes a protein of interest, (4) an antibody or other protein that recognizes a specific tag fused to the protein of interest, or (5) any other means of specifically recognizing a protein of interest in situ. The base editor may be (1) an APOBEC family base editor, (2) an ADAR family base editor, or (3) any base editor that alters specific bases of DNA or RNA to a different base, or any modification of the nucleoside (such as methylation of the base) with this alteration being detectable by later DNA sequencing. Together, the combination of recognition module with base editor is termed the “recognition-editing system” herein. An exemplary fusion polypeptide of a recognition-editing system described herein comprises an amino acid sequence as set forth in SEQ ID NO: 1:

(SEQ ID NO: 1)
MKSSHHHHHHGSSMKIEEGKLVIWINGDKGYNGLAEVGKKFEKDTGIKVT
VEHPDKLEEKFPQVAATGDGPDIIFWAHDRFGGYAQSGLLAEITPDKAFQ
DKLYPFTWDAVRYNGKLIAYPIAVEALSLIYNKDLLPNPPKTWEEIPALD
KELKAKGKSALMENLQEPYFTWPLIAADGGYAFKYENGKYDIKDVGVDNA
GAKAGLTFLVDLIKNKHMNADTDYSIAEAAFNKGETAMTINGPWAWSNID
TSKVNYGVTVLPTFKGQPSKPFVGVLSAGINAASPNKELAKEFLENYLLT
DEGLEAVNKDKPLGAVALKSYEEELAKDPRIAATMENAQKGEIMPNIPQM
SAFWYAVRTAVINAASGRQTVDEALKDAQTNSSSNNNNNNNNNNLGIEEN
LYFQSNAGGSSETGPVAVDPTLRRRIEPHEFEVFFDPRELRKETCLLYEI
NWGGRHSIWRHTSQNTNKHVEVNFIEKFTTERYFCPNTRCSITWFLSWSP
CGECSRAITEFLSRYPHVTLFIYIARLYHHADPRNRQGLRDLISSGVTIQ
IMTEQESGYCWRNFVNYSPSNEAHWPRYPHLWVRLYVLELYCIILGLPPC
LNILRRKQPQLTFFTIALQSCHYQRLPPHILWATGLKGSGTSGAGSATAG
SGAGGQVQLVESGGGLVQAGDSLRLSCVASGRSLDGATMRWYRQAPGKER
EFVAGIFWDEIGTEYADTAKGRFTISRDNAKNTIYLQMTNLRSEDTAMYY
CNGLVFGGEYWGQGTQVTVSSGGC.

In some embodiments, the engineered, non-naturally occurring recognition-editing system of the present disclosure comprises a fusion polypeptide. In some embodiments, the recognition-editing system of the present disclosure comprises a fusion polypeptide comprising an amino acid sequence that is at least 70%, at least 75%, at least 80%, at least 85%, at least 90%, at least 95%, at least 99%, or 100% identical to SEQ ID NO: 1.

Provided herein is a fusion polypeptide comprising (a) a protein-specific recognition module; and (b) a nucleic acid base editor domain. In some embodiments, the nucleic acid base editor domain is capable of (i) altering one or more specific bases of a nucleic acid to a different base; or (ii) modifying a specific nucleoside of a nucleic acid in a manner that is detectable by nucleic acid sequencing.

In some embodiments, the protein-specific recognition module is an antibody or an antigen binding fragment thereof, a single-domain antibody or an antigen binding fragment thereof, or a nanobody or an antigen binding fragment thereof.

In some embodiments, the protein-specific recognition module specifically binds to a target protein. In some embodiments, the protein-specific recognition module comprises a primary antibody or an antigen binding fragment thereof that specifically binds to a target protein. In some embodiments, the protein-specific recognition module comprises a single-domain antibody or an antigen binding fragment thereof that specifically binds to a target protein. In some embodiments, the protein-specific recognition module comprises a nanobody or an antigen binding fragment thereof that specifically binds to a target protein. In some embodiments, the target protein is an RNA-binding protein (RBP).

In some embodiments, the protein-specific recognition module specifically binds to a supplementary recognition component. In some embodiments, the protein-specific recognition module comprises a primary antibody or an antigen binding fragment thereof that specifically binds to a supplementary recognition component. In some embodiments, the protein-specific recognition module comprises a single-domain antibody or an antigen binding fragment thereof that specifically binds to a supplementary recognition component.

In some embodiments, the protein-specific recognition module comprises a nanobody or an antigen binding fragment thereof that specifically binds to a supplementary recognition component. In some embodiments, the supplementary recognition component is a primary antibody that specifically binds to a target protein of interest, such as an RBP of interest.

In some embodiments, the protein-specific recognition module specifically binds to an Immunoglobulin (e.g., Immunoglobulin G (IgG)). In some embodiments, the protein-specific recognition module specifically binds to rabbit Immunoglobulin G (IgG) or mouse IgG. In some embodiments, the nanobody specifically binds to rabbit Immunoglobulin G (IgG). In some embodiments, the nanobody specifically binds to mouse Immunoglobulin G (IgG).

In some embodiments, the nucleic acid base editor domain is capable of (i) altering one or more specific bases of a nucleic acid to a different base; or (ii) modifying a specific nucleoside of a nucleic acid in a manner that is detectable by nucleic acid sequencing. In some embodiments, the nucleic acid base editor domain comprises a deaminase enzyme capable of catalyzing the conversion of a nucleoside to Uracil (U) or Inosine (I) in RNA.

In some embodiments, the nucleic acid base editor domain comprises a cytosine deaminase capable of catalyzing C-to-U conversion in RNA. Non-limiting examples of cytosine deaminases include APOBEC family cytosine deaminases, such as APOBEC1 (NCBI Reference Sequence: NP_001291495.1, “C->U-editing enzyme APOBEC-1 isoform a [Homo sapiens],” APOBEC2 (NCBI Reference Sequence: NP_006780.1, “C->U-editing enzyme APOBEC-2 [Homo sapiens]”), APOBEC3A (NCBI Reference Sequence: NP_001257335.1, “DNA dC->dU-editing enzyme APOBEC-3A isoform b [Homo sapiens]”), APOBEC3B (NCBI Reference Sequence: NP_001257340.2, “DNA dC->dU-editing enzyme APOBEC-3B isoform b [Homo sapiens]”), APOBEC3C (NCBI Reference Sequence: NP_055323.2, “DNA dC->dU-editing enzyme APOBEC-3C [Homo sapiens]”), APOBEC3C (also known as APOBEC3E) (NCBI Reference Sequence: NP_689639.2, “DNA dC->dU-editing enzyme APOBEC-3D isoform 1 [Homo sapiens]”), APOBEC3F (NCBI Reference Sequence: NP_001006667.1, “DNA dC->dU-editing enzyme APOBEC-3F isoform b [Homo sapiens]”), APOBEC3G (NCBI Reference Sequence: NP_068594.1, “DNA dC->dU-editing enzyme APOBEC-3G isoform 1 [Homo sapiens]”), APOBEC3H (NCBI Reference Sequence: NP_001159474.2, “DNA dC->dU-editing enzyme APOBEC-3H isoform SV-182 [Homo sapiens]”), APOBEC4 (NCBI Reference Sequence: NP_982279.1, “putative C->U-editing enzyme APOBEC-4 [Homo sapiens]”), and Activation-induced (cytidine) deaminase (AID) (NCBI Reference Sequence: NP_001317272.1, “single-stranded DNA cytosine deaminase isoform 2 [Homo sapiens]”). Members of the APOBEC family of proteins perform mRNA modifications by deaminating cytidine bases to uracil. The N-terminal domain of APOBEC like proteins is the catalytic domain, while the C-terminal domain is a pseudocatalytic domain. The catalytic domain is a zinc dependent cytidine deaminase domain and is essential for cytidine deamination. The positively charged zinc ion in the catalytic domain attracts to the partial-negative charge of RNA.

In some embodiments, the nucleic acid base editor domain comprises an adenosine deaminase capable of catalyzing Adenosine (A) to Inosine (I) conversion in RNA. Non-limiting examples of adenosine deaminases include ADAR family adenosine deaminases, such as Non-limiting examples of members of the ADAR protein family include ADAR1 (NCBI Reference Sequence: NP_001020278.1, “double-stranded RNA-specific adenosine deaminase isoform d [Homo sapiens]”), ADARB1 (ADAR2) (NCBI Reference Sequence: NP_001103.1, “double-stranded RNA-specific editase 1 isoform 1 [Homo sapiens]”), and ADARB2 (ADAR3) (NCBI Reference Sequence: NP_061172.1, “double-stranded RNA-specific editase B2 [Homo sapiens]”). ADAR enzymes bind to double-stranded RNA (dsRNA) and convert adenosine to inosine (hypoxanthine) by deamination. ADAR proteins act post-transcriptionally, changing the nucleotide content of RNA. The conversion from adenosine to inosine (A to I) in the RNA disrupts the normal A:U pairing, destabilizing the RNA. Inosine is structurally similar to guanine (G) which leads to inosine to cytosine (I:C) binding. Inosine typically mimics guanosine during translation but can also bind to uracil, cytosine, and adenosine, though it is not favored.

In some embodiments, the nucleic acid base editor domain comprises an APOBEC family base editor or an ADAR family base editor. In some embodiments, the nucleic acid base editor domain comprises an APOBEC1 enzyme. The APOBEC1 enzyme can be a mammalian APOBEC1 enzyme, such as a rat APOBEC1 enzyme, a mouse APOBEC1 enzyme, a human APOBEC1 enzyme, or another mammalian APOBEC1 enzyme. In some embodiments, the fusion polypeptide comprises an amino acid sequence that is at least 70%, at least 75%, at least 80%, at least 85%, at least 90%, at least 95%, at least 99%, or 100% identical to SEQ ID NO: 1. In some embodiments, the fusion polypeptide comprises an amino acid sequence that comprises, consists of, or consists essentially of SEQ ID NO: 1.

In some embodiments, a fusion polypeptide comprises (a) a protein-specific recognition module; (b) a nucleic acid base editor domain; and (c) one or more linkers (e.g., peptide linkers). In some embodiments, the protein-specific recognition module and the nucleic acid base editor domain are separated by a linker. In some embodiments, the protein-specific recognition module and the nucleic acid base editor domain are connected by a linker. In some embodiments, the linker has the amino acid sequence GSGTSGAGSATAGSGAGG (SEQ ID NO: 4). Other linkers that may be used include, but are not limited to: repeats of glycine, repeats of glycine and serine, and repeats of alanine. In general, linkers that can connect one or more domains of a fusion polypeptide for the purposes of expression in a cell will comprise about 3 to about 20 amino acids. For example, a peptide linker may be 2, 3, 4, 5, 6, 7, 8, 9, 10, 11, 12, 13, 14, 15, 16, 17, 18, 19, or 20 amino acids.

In some embodiments, an engineered, non-naturally occurring recognition editing system comprises a supplementary recognition component. The supplementary recognition component may be an agent that specifically binds to a target protein of interest, but is not fused to a base editor domain. For example, the supplementary recognition component can be or comprise a primary antibody or fragment thereof that specifically binds to the target protein.

In some embodiments, an engineered, non-naturally occurring recognition editing system comprises (1) a fusion polypeptide comprising a protein-specific recognition module and a base editor domain; and (2) a supplementary recognition component. In some embodiments, the protein-specific recognition module specifically binds to the supplementary recognition component, and the supplementary recognition component specifically binds to a protein of interest. In some embodiments, the fusion polypeptide of the recognition-editing system may include a nucleic acid base editor domain fused to a secondary antibody or fragment thereof, or a nanobody or an antigen binding fragment thereof, that is capable of binding to a primary antibody or fragment thereof of the supplementary recognition component. In this way, the supplementary recognition component acts as an intermediate protein bound to a protein of interest.

In some embodiments, an engineered, non-naturally occurring recognition-editing system comprising a fusion polypeptide comprises (a) a protein-specific recognition module, wherein the module specifically recognizes a target protein; and (b) a nucleic acid base editor domain, wherein the nucleic acid base editor domain is capable of (i) altering one or more specific bases of a nucleic acid to a different base; or (ii) modifying a specific nucleoside of a nucleic acid in a manner that is detectable by nucleic acid sequencing.

Provided herein are polynucleotides encoding a fusion polypeptide described herein. In some embodiments, a polynucleotide comprises a nucleic acid sequence encoding a fusion polypeptide of a recognition-editing system. In some embodiments, a polynucleotide comprises a sequence encoding an amino acid sequence that is at least 70%, at least 75%, at least 80%, at least 85%, at least 90%, at least 95%, at least 99%, or 100% identical to SEQ ID NO: 1. In some embodiments, the polynucleotide sequence is codon-optimized.

Provided herein is a vector comprising a polynucleotide of the disclosure. In some embodiments, the vector is an expression vector.

Provided herein is a cell comprising a recognition-editing system of the disclosure. Provided herein is a cell comprising a fusion polypeptide of the disclosure. Provided herein is a cell comprising a polynucleotide of the disclosure. Provided herein is a cell comprising a vector of the disclosure. In some embodiments, the cell is a human cell. Non-limiting examples of human cells include HEK293T cells and HeLa cells. In some embodiments, the cell is a HEK293T cell.

### Methods for In Situ Protein-Nucleic Acid Profiling

Provided herein are methods for in situ profiling of protein-nucleic acid interactions. Also provided herein are methods for identifying one or more nucleic acid binding site of a target protein or target protein complex in a biological sample. In some embodiments, the nucleic acid binding site is a nucleic acid binding site in DNA. In some embodiments, the nucleic acid binding site is a nucleic acid binding site in RNA.

In some embodiments, provided is a method of identifying one or more nucleic acid binding site of a target protein or target protein complex in a biological sample, comprising contacting the biological sample with an engineered, non-naturally occurring recognition-editing system. In some embodiments, the engineered, non-naturally occurring recognition-editing system comprises a fusion polypeptide comprising: (a) a protein-specific recognition module, wherein the module specifically recognizes a target protein; and (b) a nucleic acid base editor domain, wherein the nucleic acid base editor domain is capable of (i) altering one or more specific bases of a nucleic acid to a different base; or (ii) modifying a specific nucleoside of a nucleic acid in a manner that is detectable by nucleic acid sequencing. In some embodiments, the engineered, non-naturally occurring recognition-editing system further comprises a supplementary recognition component.

Contacting the biological sample with the engineered, non-naturally occurring recognition-editing system can include contacting the biological sample with a fusion polypeptide comprising: (a) a protein-specific recognition module, wherein the module specifically recognizes a target protein; and (b) a nucleic acid base editor domain, wherein the nucleic acid base editor domain is capable of (i) altering one or more specific bases of a nucleic acid to a different base; or (ii) modifying a specific nucleoside of a nucleic acid in a manner that is detectable by nucleic acid sequencing. Contacting the biological sample with the engineered, non-naturally occurring recognition-editing system can include contacting the biological sample with a supplemental recognition component. In some embodiments, contacting the biological sample with the engineered, non-naturally occurring recognition-editing system comprises contacting the biological sample with the fusion polypeptide. In some embodiments, contacting the biological sample with the engineered, non-naturally occurring recognition-editing system comprises contacting the biological sample with both the fusion polypeptide and the supplementary recognition component. The fusion polypeptide and the supplementary recognition component can contact the biological sample simultaneously or sequentially.

Contacting the biological sample with the engineered, non-naturally occurring recognition-editing system can be performed for a time sufficient to permit localization of the recognition-editing system (e.g., the fusion polypeptide, the supplementary recognition component, or both) to a target protein. For example, contacting the biological sample with the engineered, non-naturally occurring recognition-editing system can include contacting the biological sample with the fusion polypeptide for at least 15 minutes, at least 20 minutes, at least 30 minutes, at least 1 hour, at least 2 hours, or at least 3 hours, at least 6 hours, at least 9 hours, at least 12 hours, at least 18 hours, or at least 24 hours. In some embodiments, contacting the biological sample with the fusion polypeptide is performed for about 1 hour.

In some embodiments, following contacting the biological sample with the fusion polypeptide, the biological sample is washed with a buffer solution to reduce non-specific binding of the protein-specific recognition module of the fusion polypeptide within the biological sample.

In some embodiments, before contacting the biological sample with the fusion polypeptide, the biological sample is contacted with a supplementary recognition component, such as a primary antibody that specifically binds to a target protein of interest (e.g., an RNA-binding protein (RBP) of interest). Contacting the biological sample with the supplementary recognition component can be performed for a time sufficient to permit localization of supplementary recognition component to the target protein. For example, contacting the biological sample with the supplementary recognition component may be performed for at least 15 minutes, at least 20 minutes, at least 30 minutes, at least 1 hour, at least 2 hours, or at least 3 hours, at least 6 hours, at least 9 hours, at least 12 hours, at least 18 hours, or at least 24 hours. In some embodiments, contacting the biological sample with a supplementary recognition component is performed for about 1 hour. In some embodiments, following contacting the biological sample with the supplementary recognition component, the biological sample is washed with buffer solution to reduce non-specific binding of the supplementary recognition component within the biological sample.

In some embodiments, after contacting the biological sample with the supplementary recognition component, and optionally after washing the biological sample with a buffer solution, the biological sample is contacted with a fusion polypeptide comprising: (a) a protein-specific recognition module, wherein the module specifically binds to the supplementary recognition component; and (b) a nucleic acid base editor domain, for a time sufficient to permit binding of the protein-specific recognition module to the supplementary recognition component. For example, contacting the biological sample with the fusion polypeptide may be performed for at least 15 minutes, at least 20 minutes, at least 30 minutes, at least 1 hour, at least 2 hours, or at least 3 hours, at least 6 hours, at least 9 hours, at least 12 hours, at least 18 hours, or at least 24 hours. In some embodiments, contacting the biological sample with fusion polypeptide is performed for about 1 hour. In some embodiments, following contacting the biological sample with the fusion polypeptide, the biological sample is washed with buffer solution to reduce non-specific binding of the protein-specific recognition module within the biological sample.

In some embodiments, the biological sample is processed before it is contacted with the recognition-editing system and/or the supplementary recognition component. In some embodiments, the biological sample is not processed before it is contacted with the recognition-editing system and/or the supplementary recognition component. In some embodiments, processing can include fixing and/or permeabilizing cells in the biological sample. The biological sample may be contained in a buffer solution. In some embodiments, processing can include semi-permanently linking the target protein to its nucleic acid binding sites.

In some embodiments, the nucleic acid base editor domain of the engineered, non-naturally occurring recognition-editing system is activated, such that it becomes catalytically active. In some embodiments, a catalytically active nucleic acid base editor domain catalyzes the conversion of Cytosine (C) to Uracil (U) in a nucleic acid (e.g., RNA). In some embodiments, a catalytically active nucleic acid base editor domain catalyzes the conversion of Adenosine (A) to Inosine (I) in a nucleic acid (e.g., RNA). In some embodiments, activating the nucleic acid base editor domain results in base edits in adjacent nucleic acids.

Activation can be accomplished by stimulating the nucleic acid base editor domain by applying physiochemical conditions sufficient for enzymatic activity, such as by providing a suitable buffer solution and a suitable operating temperature. In some embodiments, activating the nucleic acid base editor domain comprises incubating the nucleic acid base editor domain in a buffer having a temperature of about 35° C., about 36° C., about 37° C., about 38° C., or about 39° C. In some embodiments, activating the nucleic acid base editor domain comprises incubating the nucleic acid base editor domain in a buffer having a temperature of about 37° C. In some embodiments, activation is accomplished by, for example, incubating the nucleic acid base editor domain in a buffer comprising about 20 mM Tris-HCl at desired pH, about 5 mM Dithiothreitol (DTT), 2U/μL RNaseOUT, desired final concentration of NaCl). In some embodiments, activation is accomplished by, for example, incubating the nucleic acid base editor domain at about 37° C. in a buffer comprising about 20 mM Tris-HCl at desired pH, about 5 mM Dithiothreitol (DTT), 2U/μL RNaseOUT, desired final concentration of NaCl). In some embodiments, activating the nucleic acid base editor domain is performed for at least 1 hour, at least 2 hours, at least 4 hours, at least 8 hours, at least 10 hours, at least 12 hours, at least 15 hours, at least 16 hours, or at least 24 hours. In some embodiments, activating the nucleic acid base editor domain is performed for about 1 hour, about 2 hours, about 4 hours, about 8 hours, about 10 hours, about 12 hours, about 15 hours, about 16 hours, about 24 hours, about 30 hours, or about 36 hours. In some embodiments, activating the nucleic acid base editor domain is performed for about 16 hours.

In some embodiments, the method further comprises quenching or halting the base editing reaction. The base editing reaction can be quenched by, for example, incubating the nucleic acid base editor domain at about 95° C. for about 10 minutes.

In some embodiments, a method for identifying one or more nucleic acid binding site of a target protein or target protein complex in a biological sample can include extracting nucleic acids from the biological sample. In some embodiments, extracting nucleic acids from the biological sample is performed after contacting the biological sample with an engineered, non-naturally occurring recognition-editing system. In some embodiments, extracting nucleic acids from the biological sample is performed after contacting the biological sample with an engineered, non-naturally occurring recognition-editing system, and activating the nucleic acid base editor domain of the engineered, non-naturally occurring recognition-editing system.

In some embodiments, a method for identifying one or more nucleic acid binding site of a target protein or target protein complex in a biological sample can include profiling base edits by identifying one or more sequence differences in first sequence data (obtained from a biological sample contacted with the recognition-editing system) relative to second sequence data obtained from a biological sample that was not contacted with the recognition-editing system. In some embodiments, the sequence differences is a Uracil (U) in the first sequence data when a Cytosine (C) is present at the corresponding nucleic acid position in the second sequence data. In some embodiments, the position of the one or more sequence differences represents one or more nucleic acid binding site of the target protein or the target protein complex. In some embodiments, the sequence differences is an Inosine (I) in the first sequence data when an Adenosine (A) is present at the corresponding nucleic acid position in the second sequence data.

In some embodiments, the position of the one or more sequence differences represents one or more nucleic acid binding site of the target protein or the target protein complex. Profiling may include RNA sequencing analysis with a STAMP computation pipeline that maps C-to-U conversion in the transcriptome, as in Brannan et. Al., Nature Methods, 18, 57-529 (2021), which is incorporated herein by reference in its entirety. Profiling may include downstream analysis including the identification of motif, comparative analysis with enhanced crosslinking and immunoprecipitation (eCLIP) and STAMP data, and/or other downstream analyses.

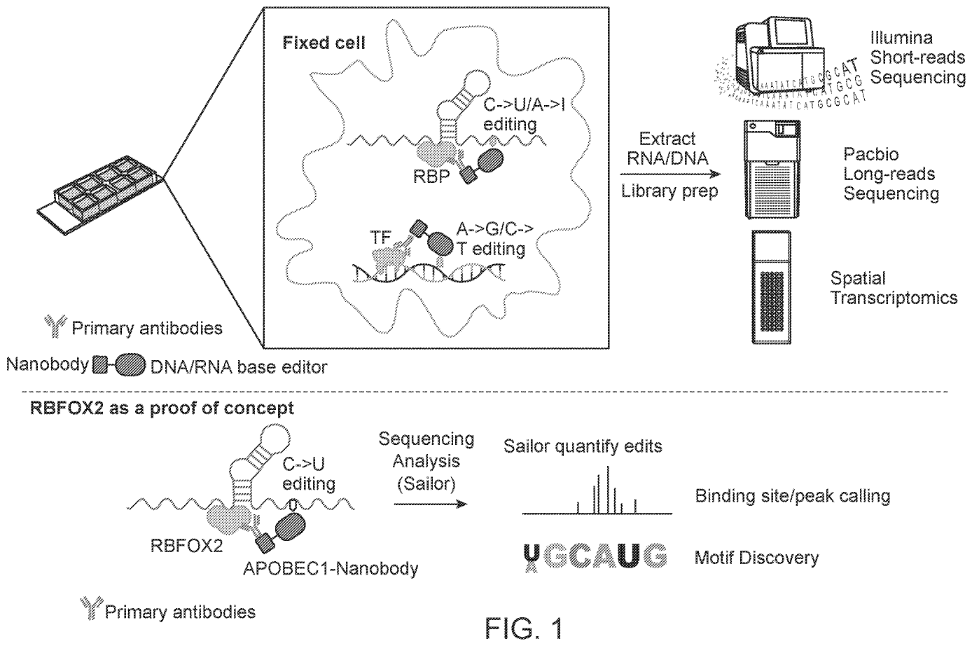

FIG. 1 depicts an exemplary design of an in situ STAMP assay as described herein. First, cells or tissues of interest can be fixed and permeabilized, semi-permanently linking each protein to its DNA or RNA binding sites. (See FIG. 1, top panel) Next, the recognition-editing system can be added such that it specifically binds to the protein of interest. For recognition-editing systems with multiple components (such as a primary antibody that recognizes the protein of interest, and an antibody-editor fusion polypeptide that specifically binds to the primary antibody), multiple washing and reagent steps may be required for this step. Following another round of washes and conversion of the buffer conditions to activate the enzymatic function of specifically-localized base editors, DNAs/RNAs in close proximity to the binding sites for the protein of interest are edited to a different base. These base changes can then be profiled by different sequencing methods, including Illumina short-read sequencing, PacBio, or Oxford Nanopore long-read sequencing, or other sequencing technologies.

The method can be combined with spatial transcriptomics methods to obtain a spatial view of DNA/RNA recognition in cells or tissues. After quantifying editing sites by analyzing and mapping sequencing data, binding sites for the protein of interest can be identified in the genome/transcriptome of the cells or tissue (See FIG. 1, bottom panel). Further, the preferred binding motif(s) of the protein of interest can be investigated through de novo motif discovery. This method can be applied to study the interactions of a single protein with nucleic acids in different tissues or cell samples, providing insights into the heterogeneity of protein-nucleic acid interactions in distinct biological contexts.

### Kits

Provided herein are kits for practicing various embodiments of the disclosure. The kit contents may be contained in a suitable container, optionally together with instructions for use in a method as disclosed herein.

Provided herein are kits comprising one or more of a recognition-editing system as described herein, a fusion polypeptide as described herein, a polynucleotide encoding a fusion polypeptide described herein, a supplementary recognition component as described herein, a reagent for fixing and/or permeabilizing cells, and a reagent for extracting nucleic acids from cells. In certain embodiments, the kit comprises packaging material and at least one container comprising one or more of a recognition-editing system as described herein, a fusion polypeptide as described herein, a polynucleotide encoding a fusion polypeptide described herein, a supplementary recognition component as described herein, a reagent for fixing and/or permeabilizing cells, and a reagent for extracting nucleic acids from cells. In certain embodiments, the kit comprises a solution with prescribed buffers and/or preservatives as described herein, optionally in an aqueous diluents, and optionally in a container, such as a vial.

### EXAMPLES

These examples are provided for illustrative purposes only and not to limit the scope of the claims provided herein.

### Example 1. Experimental Methods

With the goal of in situ identification of a protein or protein complex's binding sites on nucleic acids, the present inventors designed a recombinant fusion of APOBEC1 enzyme and a nanobody, where the nanobody could bind a rabbit-derived RBFOXII primary antibody and APOBEC1 could edit the adjacent RNAs. This enabled the identification and/or further use of RBFOXII RNA binding sites.

### Plasmid Construction and Recombinant Protein Purification

The amino acid sequence of rat APOBEC1 (NP_037039.1) was codon-optimized for expression in E. coli strain and cloned into a modified UC Berkeley Macrolab vector 2CT (Addgene #29706) to generate an N-terminal fusion to a His₆-MBP (Maltose-Binding Peptide) tag. The sequence of an engineered anti-rabbit-IgG nanobody (TP897, Addgene plasmid #104163) was cloned at the C-terminal of the APOBEC1 with an 18-aa linker (GSGTSGAGSATAGSGAGG) (SEQ ID NO: 4) in-between. Plasmid pTP1183 can be found at Addgene plasmid #104163; RRID: Addgene_104163. The His-MBP-APOBEC1-Nb (APO1-Nb) protein, having the sequence of SEQ ID NO: 1, was expressed in E. coli strain Rosetta2 PLysS (EMD Millipore) by growing cultures in 2×YT media to mid-log phase at 37° C., followed by induction with 0.25 mM IPTG at 18° C. for 16 hours supplemented with 100 μM ZnCl₂. For protein purification, cells were harvested by centrifugation, suspended in resuspension buffer (25 mM Tris-HCl pH 8.5, 1 M NaCl, 20 mM imidazole, 2 mM 0-Mercaptoethanol and 10% glycerol) with 0.1 mg/mL RNase A and lysed by sonication. Lysates were clarified by centrifugation (16,000 rpm 30 min), then the supernatant was loaded onto a Ni²⁺ affinity column (HisTrap HP, Cytiva) pre-equilibrated with the resuspension buffer. The column was washed with a buffer containing 20 mM imidazole and 500 mM NaCl, and eluted with a buffer containing 250 mM imidazole and 250 mM NaCl. The elution was buffer-exchanged into the low salt buffer (20 mM Tris pH8.5, 50 mM NaCl, 1 mM DTT and 10% glycerol) and loaded onto an anion-exchange column (HiTrap Q HP, Cytiva) following washing with a gradient of 50 mM-1 M NaCl. The eluted fractions containing the protein (evaluated with SDS-PAGE) were collected and loaded onto a size exclusion column (Superdex 200 Increase 10/300 GL, Cytiva) equilibrated with the size exclusion buffer (20 mM Tris pH7.5, 300 mM NaCl, 1 mM DTT and 10% glycerol). The peak containing the dimerized His-MBP-APOBEC1-Nb protein was collected concentrated by ultrafiltration, aliquoted and frozen at −80° C. for future use.

### In Vitro RNA Editing Assay

A 300nt RNA from the 3′-UTR of the human APP gene was in vitro-transcribed (MEGAshortscript T7 Transcription Kit, Invitrogen) and purified (Zymo RNA Clean & Concentrator). For a typical RNA editing assay, 0.1 μM RNA was incubated with 1.2 μM APO1-Nb (or other Enzyme-Nb) at 37° C. for 16 hours in RNA editing buffer (20 mM Tris-HCl at desired pH, 5 mM DTT, 2U/μL RNaseOUT, desired final concentration of NaCl). The reaction was quenched at 95° C. for 10 min. The edited RNAs were reverse-transcribed to cDNA (High-Capacity cDNA Reverse Transcription Kit, Thermo) followed by 20-cycles of PCR amplification and Sanger sequencing (Azenta).

### Cell Culture

HEK293T cells were cultured in DMEM medium (high glucose, Gibco) with 10% (v/v) heat inactivated fetal bovine serum FBS (Gibco) and 5 U/mL Penicillin-Streptomycin (Gibco) at 37° C., 5% CO2. All cell lines were maintained at 37° C. and 5% CO2 and routinely tested for the presence of mycoplasma.

### Animals

A 2-month-old female C57BL/6J wildtype mouse was used in the study. The mouse strain was obtained from The Jackson Laboratory and bred and maintained on-site. All mice were housed under specific pathogen-free conditions with a reversed 12-h light-dark cycle maintained at 23° C. with 30-70% humidity and provided with food and water ad libitum. The study was carried out in strict accordance with the recommendations in the Guide for the Care and Use of Laboratory Animals of the National Institutes of Health and approved by the Animal Care and Use Committee at University of California San Diego.

### In Situ STAMP Editing and RNA-Seq Library Preparation

Approximately 150K HEK293T cells were seeded on poly D-lysine (PDL, Sigma)-coated chamber of ibitreat 8-well slide (ibidi). Cells were cultured overnight to settle and attach to the slide.

After removing the medium and washing with PBS, the cells were fixed with 250 μL pre-chilled MeOH or 4% paraformaldehyde (PFA) for 15 min on ice. For the PFA-fixed samples, cells were permeabilized with 0.3% Triton X-100 in PBS for 20 mins following fixation. All the washings and incubations were done on ice unless otherwise stated. The Washing Buffer consists of PBS with 2 mM DTT, 0.05% Tween-20. Following 3 cycles of washing in 300 μL Washing Buffer, cells were incubated with 100 μL blocking buffer (5% BSA in Washing Buffer with 0.2U/μL RNaseOUT) for 1 hour. After blocking, the cells were incubated in 150 μL diluted primary antibodies (rabbit anti-RBFOX2, Bethyl, A300-864A; rabbit anti-TDP-43, Bethyl, A303-223A) with optimized concentration in Antibody Dilution Buffer (PBS with 1% BSA, 2 mM DTT and 0.2U/μL RNaseOUT) for 1 hour. After 3 cycles of washing in PBS-T, 100 μL 1 μg/mL APO1-Nb in dilution buffer was added for 1-hour incubation followed by 6 times of washing in PBS-T. The slides were then washed twice in RNA editing buffer (20 mM Tris pH7.5, 20 mM NaCl, 5 mM DTT and 0.2U/μL RNaseOUT) on ice. Finally, the slides were incubated at 37° C. for overnight (~16 hours). For the “Enzyme only” control, no antibody was added. For the “Buffer only” control, no antibody or enzyme was added. Three replicates were performed in three separate slides for in situ STAMP experiments, enzyme-only controls and buffer-only controls. Primary antibodies that bind to RBFOXII are known in the art. See, for example, www.abcam.com/fox2—rbm9-antibody-4g3-ab57154.html. See also www.fortislife.com/products/primary-antibodies/rabbit-anti-rbm9-antibody/BETHYL-A300-864. The amino acid sequence of the APO1-Nb fusion protein is set forth in SEQ ID NO: 1.

On the next day, cells were harvested in 200 μL Trizol. Total RNA was extracted (Zymo Direct-zol RNA Purification Kit), followed by DNA depletion (DNase I treatment and Zymo RNA Clean and Concentrator Kit) and ribosomal RNA depletion (Illumina Ribo-Zero Plus rRNA Depletion Kit). For the PFA-fixed samples, cells were reverse-crosslinked prior to RNA extraction with 72U/mL Proteinase K (NEB) in De-crosslinking Buffer (10 mM Tris pH8.0, 200 mM NaCl, 50 mM EDTA, 2% SDS) for 2 hours at 55° C. The lysate post-decrosslinking were collected in Trizol LS. The RNA-seq library was prepared following the instruction of Illumina TruSeq Stranded Total RNA Library Preparation Kit.

For in situ STAMP in mouse brain tissue, the mouse brain was collected and snap frozen. The frozen brain was then cryosectioned into 20 μm slices and fixed in pre-chilled 100% methanol for 15 min. It was then kept in methanol at −20° C. until in situ STAMP experiment. After 3 cycles of washing in PBS-T, in situ STAMP was performed similarly as described above as fixed cells.

### Immunofluorescent Imaging

Immunofluorescence (IF) was carried out similarly to the in situ STAMP protocol. All washings were done 3 times with PBS-T and the Antibody Dilution Buffer was used for all dilution series. For the primary antibody dilution series, after primary antibody (rabbit anti-RBFOX2 or rabbit anti-TDP-43) incubation and washing, the cells were incubated with 1:1000 goat anti-rabbit IgG Alexa 488 (Invitrogen) for 1 hour, followed by DAPI stain and washing before being subjected to confocal imaging. For the APO1-Nb dilution series, after incubation with a fixed optimized concentration of primary antibody, cells were incubated with a series of APO1-Nb dilutions and then 1:1000 mouse anti-MBP antibodies (Proteintech). The cells were then washed and incubated with 1:1000 goat anti-rabbit IgG Alexa 488 and 1:1000 goat anti-mouse IgG Alexa 555 (Invitrogen). Subsequently, immunofluorescence samples were imaged on an LSM-880 fluorescence confocal microscope (Zeiss) equipped with 405 nm, 488 nm, and 561 nm lasers. Images were acquired with a 20× objective for traditional confocal microscopy and 63× for Airyscan superresolution imaging.

### Analysis of Immunofluorescence Images

Confocal images were processed and analyzed with Fiji (NIH). The “Threshold” function was used to determine the average intensity of antibody or nanobody staining for the corresponding fluorescence channel; the threshold value was set to capture all cellular intensity of the antibody or nanobody. To determine the fraction of nuclear signal in each image, Applicant created masks of the whole cell using the antibody or nanobody channel and masks of the nucleus using the DAPI channel. The DAPI mask was subtracted from the whole-cell mask to create a cytoplasm-only mask of each image. The masks were subtracted from the original image to remove extraneous intensity values, and the “Threshold” function was used to determine the intensity of the remaining pixels. The total intensity (i.e. area x intensity) of the nuclear antibody signal was divided by the total intensity of the whole cell to calculate the nuclear fraction. At least 400 cells were analyzed per condition. Colocalization of Airyscan images was calculated using the “Colocalization” analysis package in Fiji; the Pearson's correlation coefficient was determined for the nanobody and antibody channels of each image. Student's t-test was performed on the Pearson's correlation coefficient of 5 images to determine the p-value.

### Illumina Sequencing and RNA Mapping

RNA-seq libraries were sequenced with single-end reads (100 nucleotides) with a typical read depth of 60M. Reads were then trimmed and filtered for repeat elements using sequences obtained from RepBase (v18.05) with STAR (2.4.0i). Reads that did not map to repeats were then mapped to the hg38 assembly with STAR (2.4.0i) for the HEK293T samples, or mapped to the mm10 assembly with STAR (2.5.2) for the mouse samples. The reads were then sorted with samtools (v1.9) and annotated against Gencode (v29).

### SAILOR Quantification of RNA Edits and FLARE Identification of Edit Clusters

The resulting BAM files were each used as inputs to SAILOR (v1.1.0) to determine C>U edit sites across the hg38 assembly. Briefly, SAILOR filters potential artifacts and known SNPs (dbSNP, v151) and returns a set of candidate edit sites and outputs the number of C>U conversions found among aligned reads. The C-to-U editing sites called with a confidence score larger than 0.5 and an editing percentage less than 0.8 were remained as the input to FLARE. The Poisson model-based FLARE accounts for background editing rates to filter false positives from truly edited regions, and scores identified clusters for use in downstream applications (https://github.com/YeoLab/FLARE). False discovery rate (FDR) threshold was set to 0.1 and the maximum merge distance was set to 15 nt. Clusters from three replicates were intersected through bedtools (2.27.1). [bedtools.intersect(wa=True, u=True, s=True)] Then, buffer-only clusters were subtracted from the intersected edit clusters and termed “confident edit clusters”.

### PacBio Long-Read Sequencing and Identification of RNA Edits

Iso-seq library preparation of RBFOX2-STAMP and enzyme-only samples was completed using the SMRTbell prep kit 3.0 (PN 102-141-700) and associated protocol. Libraries were sequenced on the Sequel II system, with one SMRT Cell allocated per sample. Following sequencing, full-length reads were obtained from consensus circular sequence (CCS) reads using the IsoSeq toolkit and standard parameters (v3.8.0). Full-length reads were aligned to the hg38 reference using pbmm2 (v1.9.0) with parameters:—preset ISOSEQ and—sort. Aligned reads were filtered for only primary mapped alignments using the SAM Flag. NanoPlot (v1.32.1) was used to assess the quality of the aligned data. Reads from both samples were clustered to resolve transcript isoforms using isoseq3 cluster (v3.8.0) with parameter:—use-qvs. Using read assignments from clustering, edits were called at the gene and isoform levels using the python implementation of pileup (pysam v0.21.0). Edits that overlapping with annotated SNP regions (dbSNP) were removed.

### STAMP Edit Cluster Evaluations

De novo motif discovery was performed using HOMER (v4.9.1) using FLARE edit clusters and a shuffled background for each UTR, CDS, intron and total genic region. A kernel density estimation (KDE) plot was used to show the distribution of the closest motif (UGCAUG or UGUGUG) distance to confident edit clusters. Edit clusters were shuffled randomly within their respective exons or introns 20 times to derive the shuffled edit clusters. The in-cell STAMP experiments compared to in situ STAMP were conducted in three replicates in HEK293T cells and computationally processed similarly. The eCLIP experiments compared to in situ STAMP were IDR (irreproducible discovery rate) peaks from RBFOX2 eCLIP in HEK293T cells (GSE77634), TDP-43 eCLIP in K562 cells (ENCODE Data Coordination Center, https://www.encodeproject.org) and RBFOX2 eCLIP in mouse whole brain tissue (GSE240326).

### Example 2. Targeting and RNA Editing In Situ by an APOBEC1-Nanobody Fusion Protein

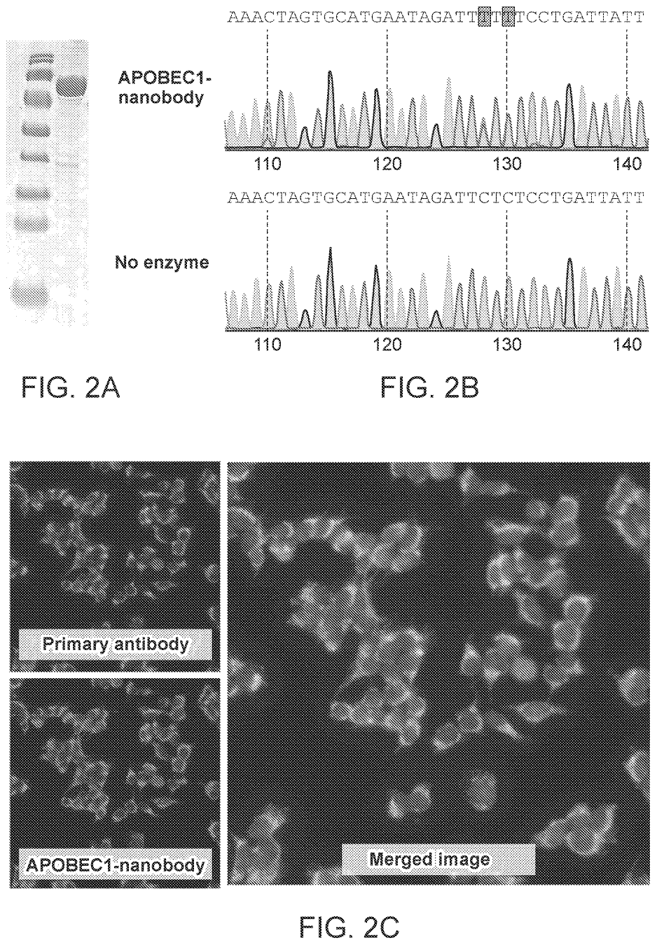

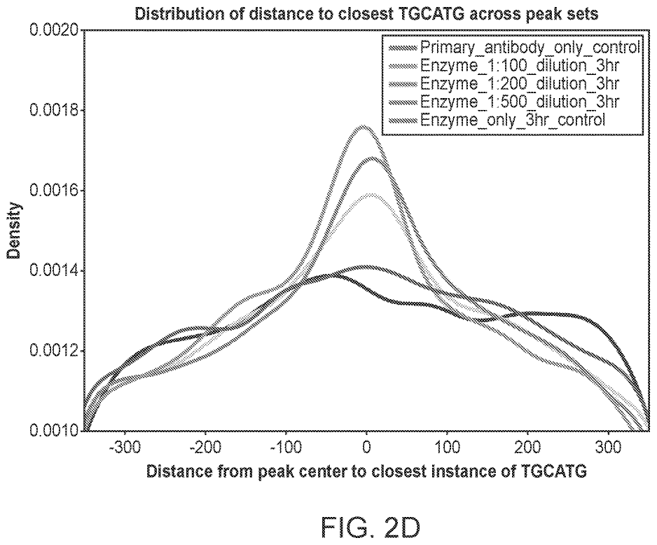

Cloning, expression, and successful purification of an APOBEC1-nanobody fusion protein comprising the amino acid sequence set forth in SEQ ID NO: 1 is shown in FIG. 2A.The fusion protein was expressed in E. coli and purified through affinity column, ion exchange column, and size exclusion column, and reached >95% purity.

As shown in FIG. 2B, in a single-gene editing assay, an in vitro-transcribed RNA was incubated with the fusion protein and Sanger sequencing reveals up high level of C-to-U editing at multiple sites on the RNA. As shown in FIG. 2C, co-localization of a primary antibody targeting RBFOXII with the recombinant APOBEC1-nanobody fusion was verified in fixed HEK293T cells by immunofluorescent staining (IF).

After in situ editing by the APOBEC1-nanobody which recognized an RBFOXII primary antibody, cells were harvested for standard RNA-sequencing. As shown in FIG. 2D, by plotting the density of the distance of the C-to-U edited peak center to the closest RBFOXII binding motif UGCAUG, the RNA editing signals driven by RBFOXII recognition could be readily observed. Thus, by analyzing the C-to-U editing signals across the transcriptome, RBFOXII binding sites were identified.

### Example 3. An APOBEC1-Nanobody Fusion Protein Mediates Targeted RNA Editing in Situ

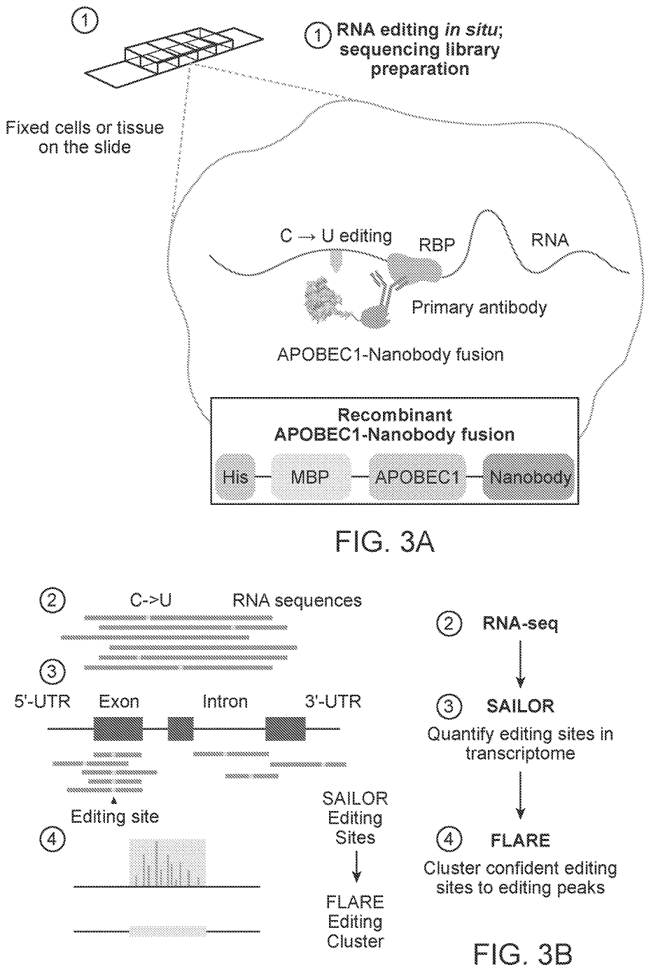

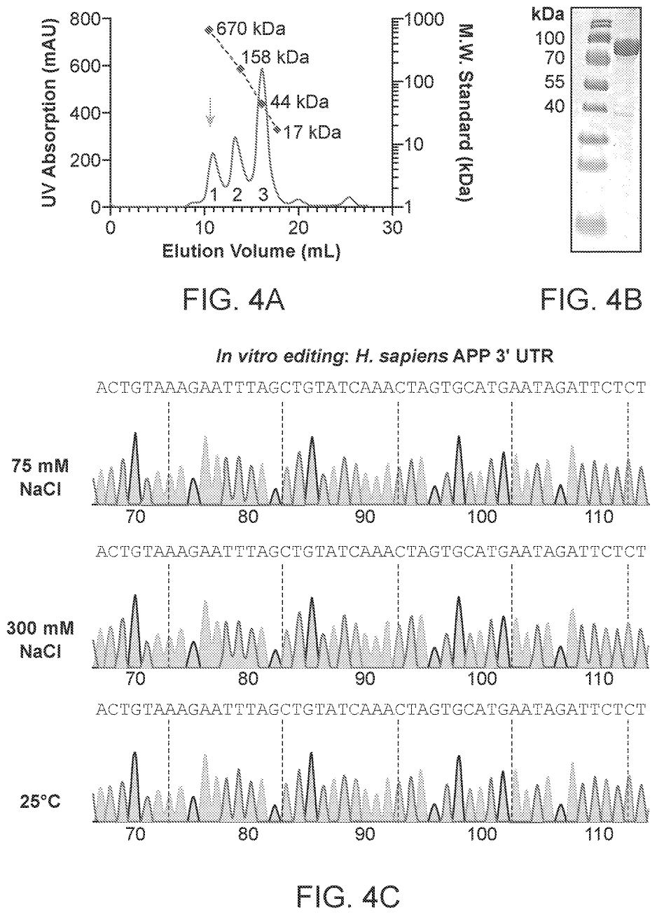

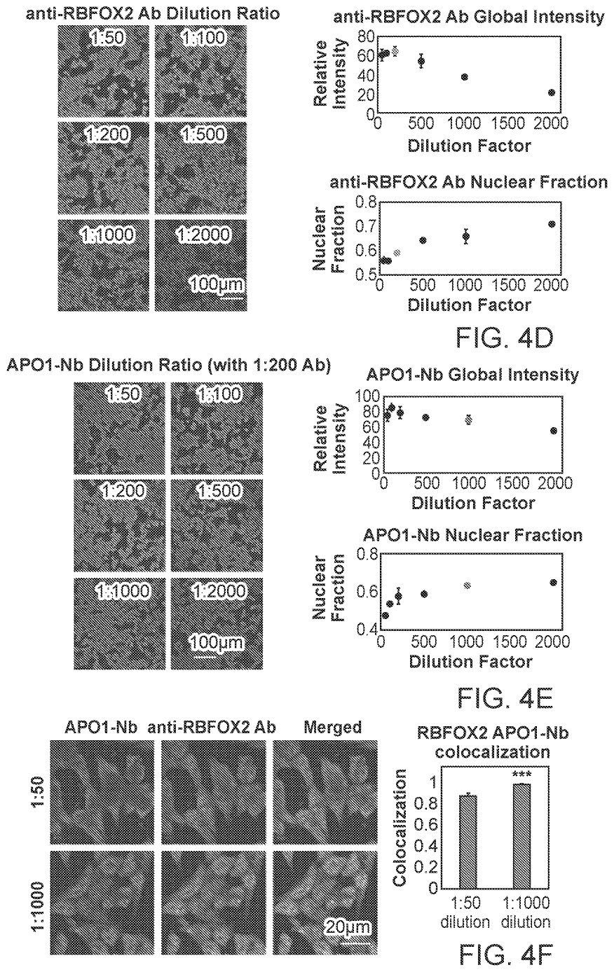

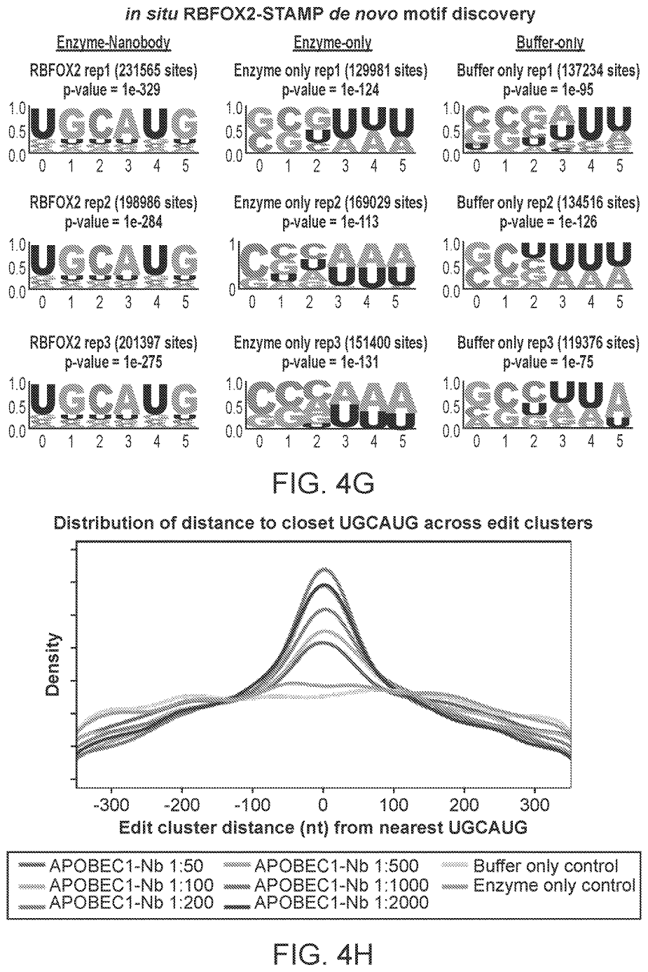

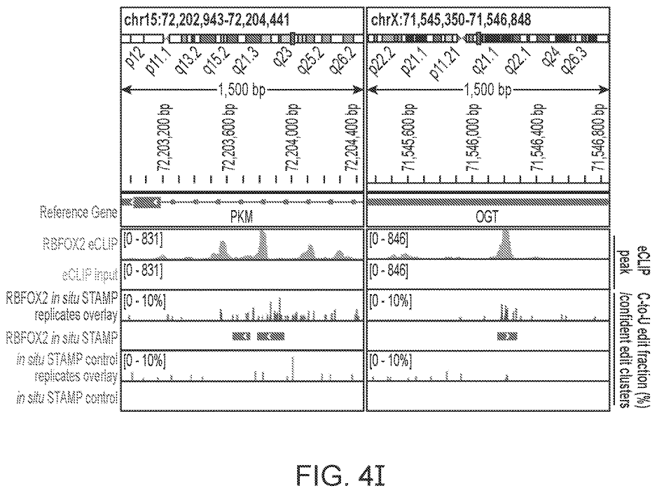

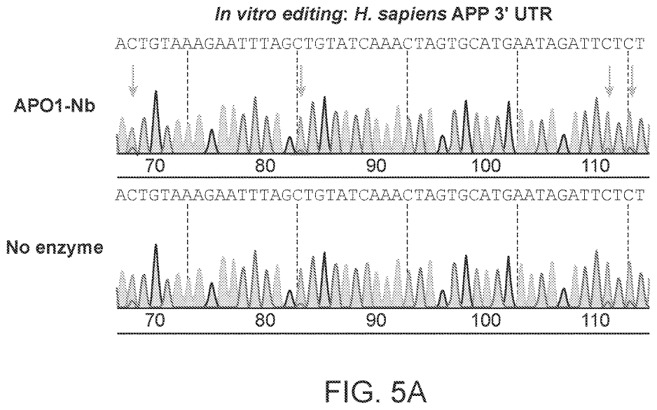

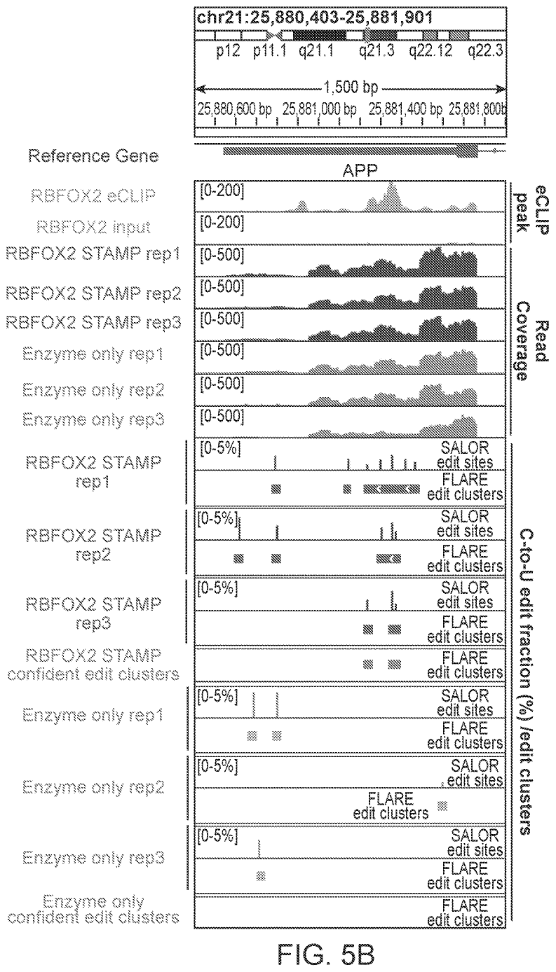

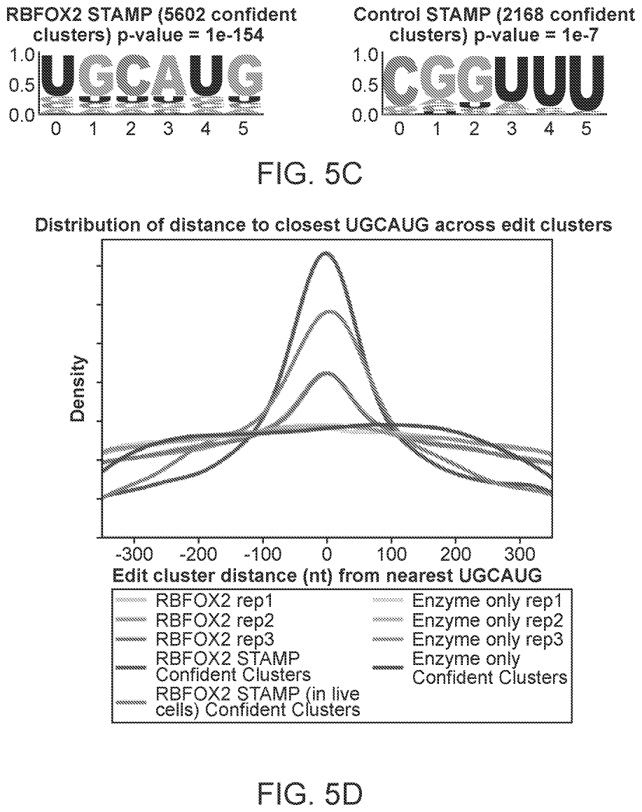

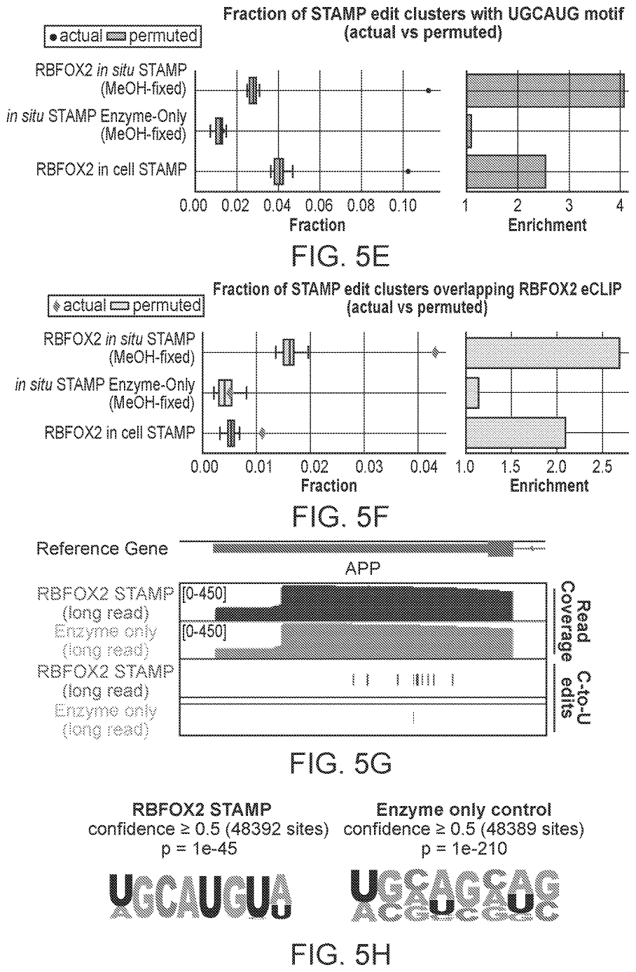

In the STAMP method, expression of a chimeric fusion of the cytosine deaminase APOBEC1 to RBPs results in specific cytosine-to-uracil (C-to-U) RNA editing in cultured cells, enabling identification of authentic RBP binding sites by high-throughput sequencing of cDNAs derived from cellular RNAs. Since APOBEC1 is also active in vitro, Applicant sought a method to circumvent the need for engineering specific RBP-APOBEC1 fusions in cells of interest. Applicant designed a fusion of APOBEC1 to a llama-derived antibody to rabbit IgG (Apo1-Nb; FIG. 3A) and expressed the fusion protein in E. coli. A multi-step purification protocol yielded a pure and homogeneous species with a molecular weight consistent with dimeric APO1-Nb (FIG. 4A-B). Applicant verified that Apo1-Nb is active by incubating the protein with an in vitro-transcribed 300 nt RNA derived from the 3′-UTR of the human APP gene, resulting in ~25% C-to-U editing at multiple sites as assayed by Sanger sequencing (FIG. 5A). Notably, Applicant observed no editing in high-salt buffers (75 mM or 300 mM NaCl) or at low temperature (25° C.), permitting precise control of the editing reaction and minimization of non-specific RNA editing (FIG. 4C). Applicant used immunostaining to show that Apo1-Nb was recruited to the nucleus of methanol-fixed HEK293T cells in the presence of a rabbit-derived antibody against the RBP RBFOX2 (FIG. 4D-F). Using this system, Applicant systematically varied the concentrations of both the anti-RBFOX2 primary antibody and Apo1-Nb to optimize specificity, as measured by the nuclear fraction of anti-RBFOX2 antibody localization and the co-localization of the anti-RBFOX2 antibody and Apo1-Nb. Applicant chose a 1:200 dilution of anti-RBFOX2 Ab and a 1:1000 dilution of APO1-Nb to maximize signal intensity (Global Intensity) while maintaining specificity (Nuclear Fraction). Thus, Apo1-Nb is an active RNA base editor that can be specifically recruited to sites of interest by primary antibodies in fixed cells.

Applicant next carried out in situ RNA editing in methanol-fixed HEK293T cells. Applicant fixed and permeabilized ~150,000 HEK293T cells on a slide, blocked with BSA, and incubated the rabbit-derived anti-RBFOX2 antibody with the cells. After washing, Applicant added APO1-Nb on ice in PBS-T buffer, where the temperature and high salt concentration prevented non-specific RNA editing. Slides were then washed and incubated at 37° C. in an RNA editing buffer containing 20 mM NaCl. After an overnight incubation, Applicant extracted RNA, depleted samples of ribosomal RNAs, and generated RNA-seq libraries. Applicant sequenced each sample to a depth of 60 million reads/replicate. Multiple control samples were incorporated, including “Enzyme only” which lacked the anti-RBFOX2 primary antibody, and “Buffer only” which lacked both the anti-RBFOX2 antibody and Apo1-Nb.

Applicant modified our established STAMP computational pipeline to identify clusters of C-to-U editing sites representing RBP-RNA interaction sites in the transcriptome. In brief, C-to-U editing events were identified by transcriptomic mapping and quantification through a modified SAILOR pipeline, followed by editing cluster identification through FLARE (FLagging Areas of RNA-editing Enrichment) (FIG. 3B). FLARE builds on the outputs of the SAILOR edit site discovery tool to identify regions statistically enriched for RNA editing (See Example 1). Based on our in vitro RNA editing assay, Applicant reasoned that in situ RNA editing efficiency is likely low and dependent on local sequence context, resulting in high noise especially when the sequencing read coverage on a given RNA is low. Therefore, Applicant took a conservative approach to cluster identification. Applicant filtered out editing sites with a SAILOR score <0.5 or an editing fraction >80% before proceeding to FLARE, which retained 75%-80% SAILOR C-to-U edits sites. With ~60 million reads per replicate, the pipeline typically identified around 200,000 edit clusters across the transcriptome in each replicate for in situ RBFOX2-STAMP, while roughly 120,000 edit clusters were determined in both the enzyme-only controls and buffer-only controls. Sites called in buffer-only controls represent false editing sites introduced by RNA damage, reverse transcription errors, or PCR errors during the experimental protocol and library preparation. Nonetheless, all three replicates of in situ RBFOX2-STAMP showed clustered C-to-U editing sites at the 3′-untranslated region (3′-UTR) of the known RBFOX2 target mRNA APP, whereas enzyme-only controls showed few or no editing sites on this mRNA. The typical C-to-U editing rate is 3-5% at certain C locations (FIG. 5B). The canonical RBFOX2 binding motif (U)GCAUG was enriched in de novo motif discovery in all replicates of RBFOX2 in situ STAMP (FIG. 4G), demonstrating the sensitivity and specificity of this method for discovering authentic RBP binding sites.

Applicant further investigated the effect of APO1-Nb concentration on the specificity of RBFOX2 in situ STAMP by measuring enrichment of the canonical RBFOX2 motif (UGCAUG) in edit clusters (FIG. 411). The best enrichment of proximal UGCAUG in edit clusters was observed in 1:1000 APO1-Nb dilution ratio (corresponding to a final concentration of 1 μg/mL). Higher or lower concentrations of APO1-Nb led to reduced RBP probing specificity, due to either insufficient Apo1-Nb concentration leading to sub-optimal editing efficiency, or oversaturated APO1-Nb leading to increased non-specific. This result agrees with our immunofluorescent staining observations (FIG. 4D-F), and Applicant therefore concluded that 1 μg/mL APO1-Nb is optimal for RBFOX2 in situ STAMP.

Since the number of edit clusters identified in both buffer-only and enzyme-only negative controls were nontrivial, Applicant applied further background noise elimination strategies. Applicant first intersected the edit clusters across replicates, retaining only edit clusters that appeared in all three experimental replicates. To reduce experimental noise introduced by endogenous C-to-U editing in cells, edit clusters identified in buffer-only controls were removed from all intersected STAMP edit clusters. These two steps removed 97-98% of edit clusters identified in only one or two individual replicates, and the remaining edit clusters were termed “confident edit clusters” and used for further analysis. 5602 confident edit clusters were obtained in RBFOX2 in situ STAMP in methanol-fixed HEK293T cells, while 2168 confident edit clusters were determined in enzyme-only controls. The canonical RBFOX2 motif was further enriched at RBFOX2 in situ STAMP confident edit clusters (p=1e-154), whereas the RBFOX2 motif was not enriched in both enzyme-only and buffer-only controls (FIG. 5C). Applicant found that confident edit clusters obtained after intersecting three replicates showed higher enrichment of UGCAUG compared to edit clusters identified in individual replicates, demonstrating the enhanced specificity of RBFOX2 target RNA identification after these noise-reduction strategies (FIG. 5D).

Further examination of the confident edit clusters showcased the accuracy of RBP binding sites discovered by in situ STAMP. Applicant found that around 10% of confident RBFOX2-STAMP edit clusters contain the UGCAUG motif with a 4-fold enrichment over the randomly shuffled (permuted) size-matched clusters within the same regions (FIG. 5E), which was comparable to in-cell RBFOX2-STAMP. In contrast, confident edit clusters in enzyme-only controls showed no enrichment of the UGCAUG motif over permuted clusters, indicating the randomness of editing in the absence of the anti-RBFOX2 primary antibody. Reproducible peaks from irreproducible discovery rate (IDR) analysis of HEK293T RBFOX2 eCLIP data serve as empirically defined RBFOX2 sites and were compared to in situ RBFOX2-STAMP. Applicant observed that the fraction of in situ RBFOX2-STAMP confident edit clusters overlapping with RBFOX2-eCLIP IDR peaks was enriched (>2.5 fold) compared to permuted clusters, indicating that in situ STAMP identifies authentic RBFOX2 binding sites (FIG. 5F). Examination of individual edit clusters of in situ STAMP revealed their proximity and concurrence with eCLIP peaks (FIG. 4I).

A key advantage of the STAMP method lies in its ability to distinguish between mRNA isoforms, due to the non-RNA-fragmenting protocol. To examine whether in situ STAMP is compatible with long-read sequencing, Applicant directly sequenced libraries from RBFOX2 in situ STAMP and enzyme-only controls using the PacBio long-read sequencing platform. Long-read sequencing of RBFOX2 in situ STAMP revealed enriched edits in the 3′ UTR of the APP gene compared to controls, and these edits overlapped with both RBFOX2-eCLIP peaks and short-read (Illumina) in situ STAMP edit clusters (FIG. 5G). De novo motif analysis revealed enrichment of the canonical RBFOX2 binding motif UGCAUG among edit sites with confidence >0.5 (48,392 sites, p=1e-45) (FIG. 511). Collectively, Applicant established an optimal working protocol for in situ STAMP by APO1-Nb RNA editing in MeOH-fixed HEK293T cells. This enabled the successful identification of RBFOX2 binding sites in situ with both short-read and long-read sequencing platforms.

### Example 4. In Situ STAMP Identifies RBP Binding Sites Across a Range of Sequencing Depths

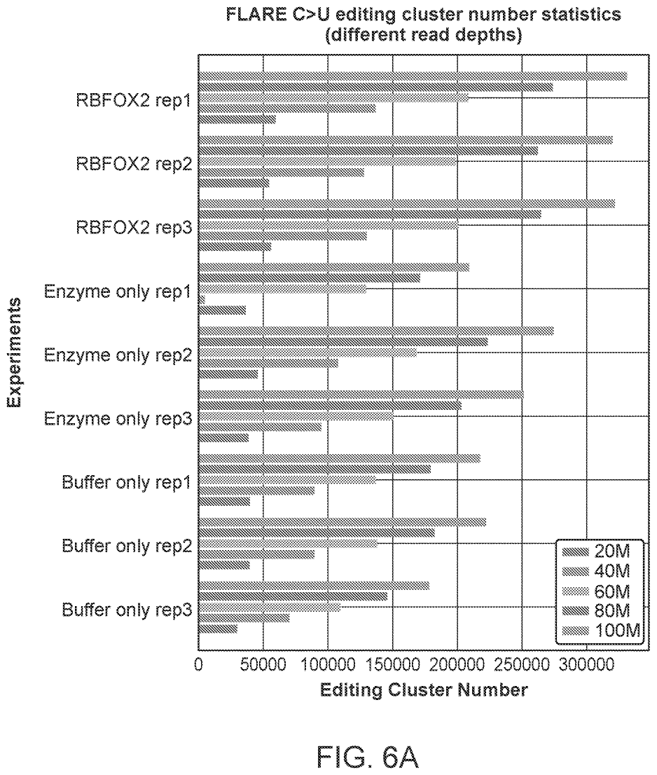

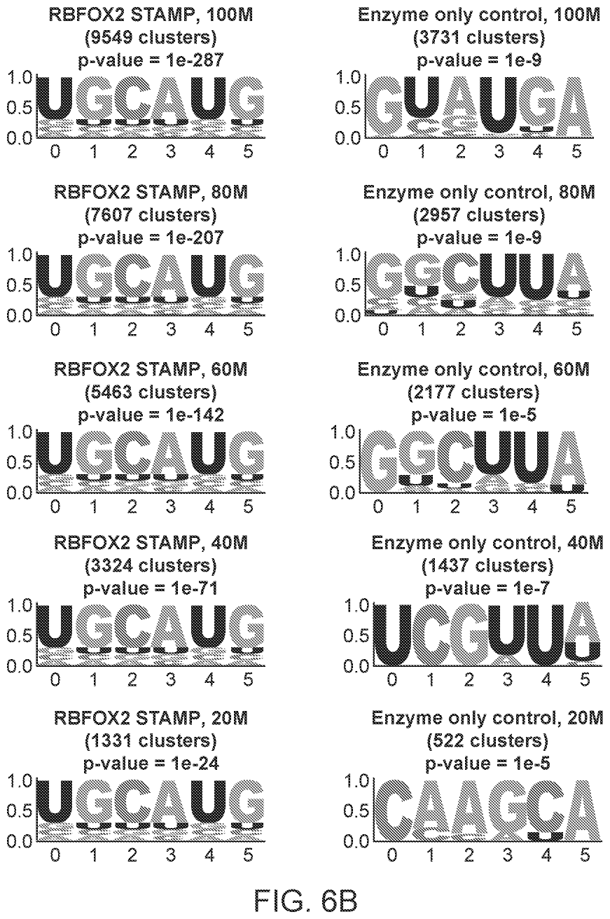

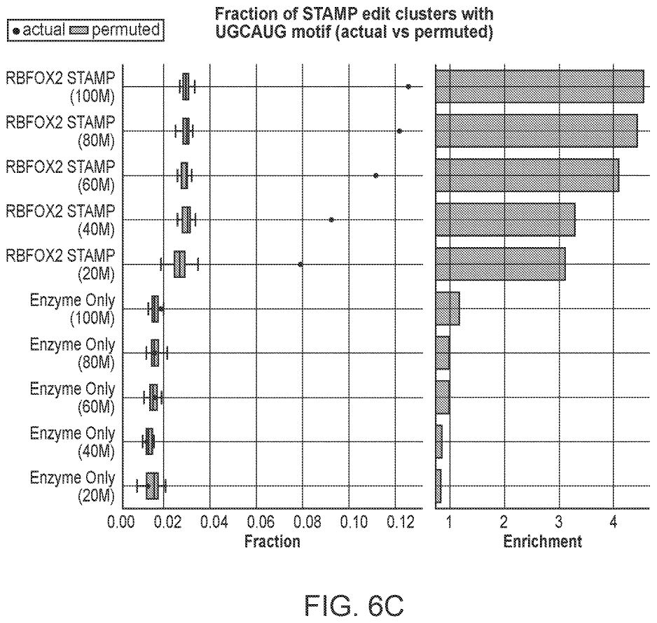

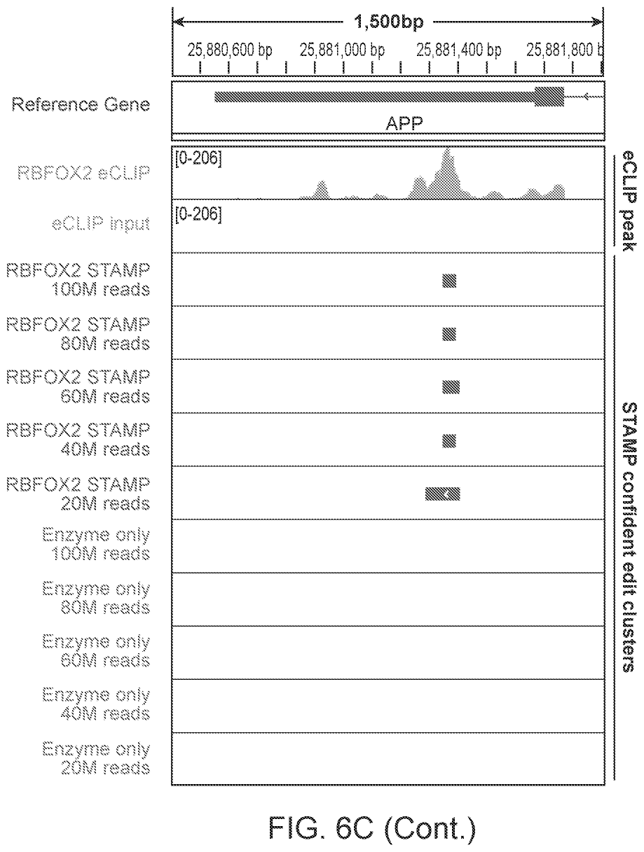

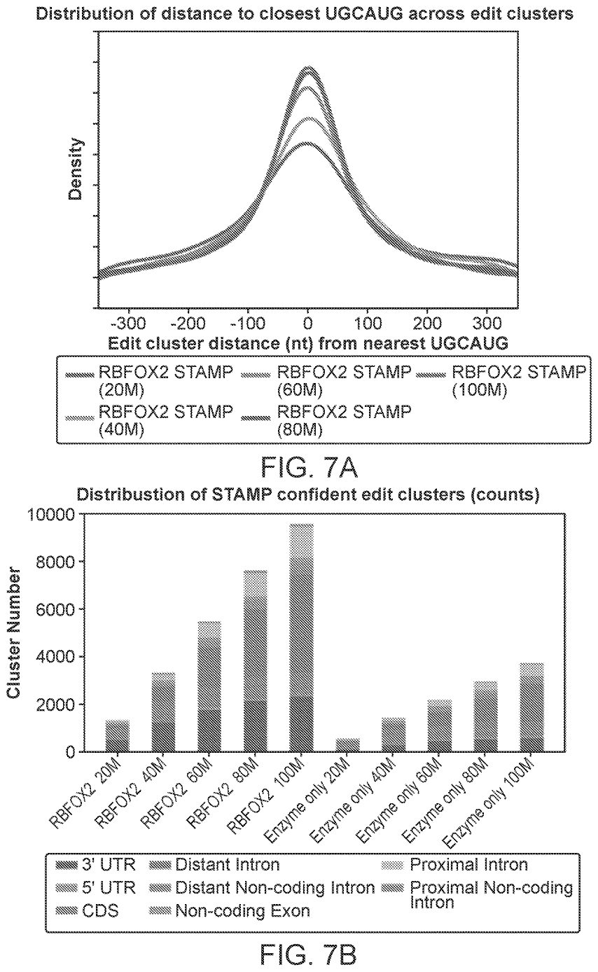

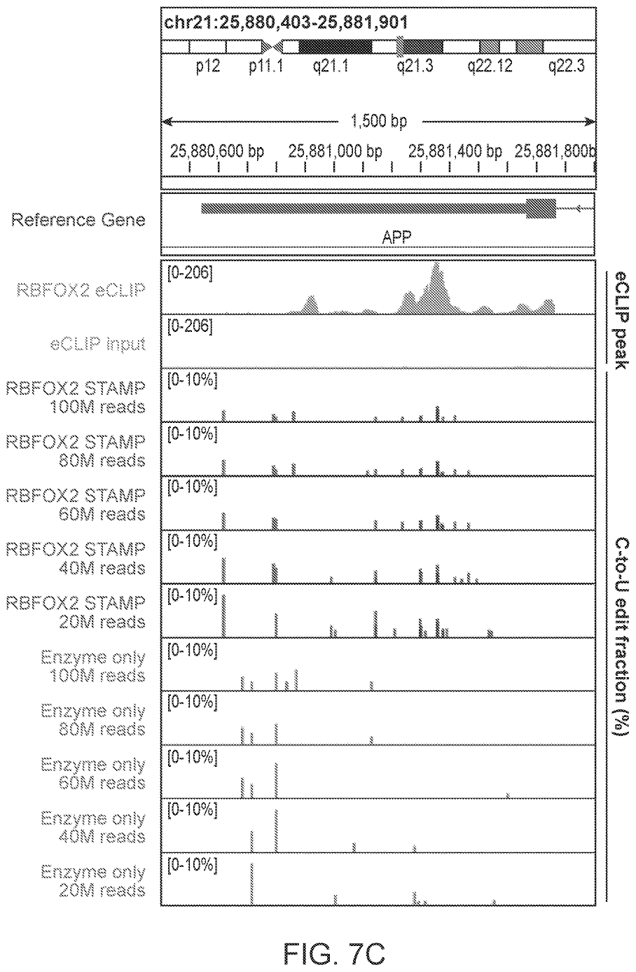

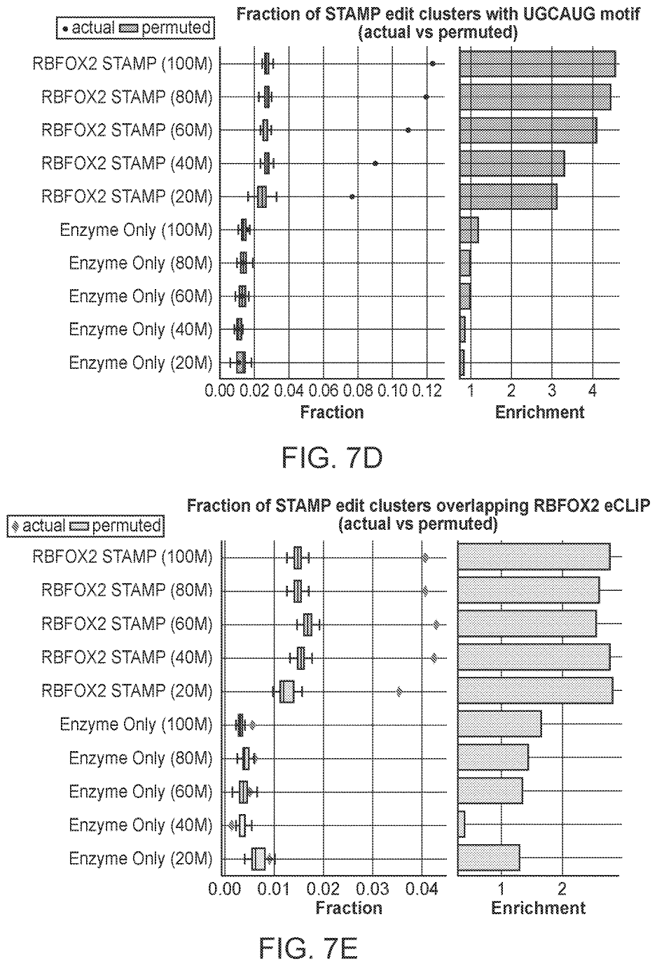

Applicant hypothesized that the accuracy of C-to-U edit cluster calls is linked to the sequencing read depth of the RNA in question, potentially making it challenging to identify RBP-RNA interactions on low-level transcripts or in intronic regions that are typically less covered, particularly in our in situ approach. To examine the effect of sequencing depth on in situ STAMP results and uncover intronic binding sites of RBFOX2, Applicant sequenced libraries from in situ RBFOX2-STAMP and controls from methanol-fixed HEK293T cells to roughly 100 million (100M) reads per replicate, then randomly down-sampled each dataset to 80, 60, 40, and 20 million reads. The number of C-to-U edit clusters determined by FLARE was proportionally associated with sequencing depth in each sample (FIG. 6A). More confident edit clusters were identified with higher sequencing depth, but de novo motif analysis could identify the canonical RBFOX2 motif in all samples regardless of sequencing depth (FIG. 6B). Enrichment of the UGCAUG motif was generally higher in deeper-sequenced data, although the difference between 100M reads and 80M reads was negligible (FIG. 7A and FIG. 7D). As expected, increased sequencing depth resulted in an increased count and proportion of identified intronic edit clusters (FIG. 7B). With 100M reads, 61.5% (5938 counts) of the confident edit clusters fell into intronic regions, whereas only 21.5% (285 counts) of the confident clusters were intronic with 20M reads. At the level of individual sites, deeper sequencing depth effectively reduced some inflated C-to-U edit fractions and thereby reduced the noise arising from low read coverage (FIG. 7C). Nonetheless, both the overall SAILOR-quantified edit site patterns and the FLARE confident edit clusters remained similar across all samples from 20M to 100M reads (FIG. 7C and FIG. 6C). Irrespective of sequencing depth, in situ STAMP showed a similar level of concordance with eCLIP IDR peaks (FIG. 7E). Overall, in situ STAMP demonstrates effective identification of RBP binding sites even with sequencing depth as low as 20M reads. For targets such as intronic sites or low-level transcripts, deeper sequencing depths are helpful; however, exceeding 100M reads per sample is unnecessary.

### Example 5. In Situ STAMP Identifies Authentic TDP-43 Binding Sites Through Confident C-to-U Edit Clusters

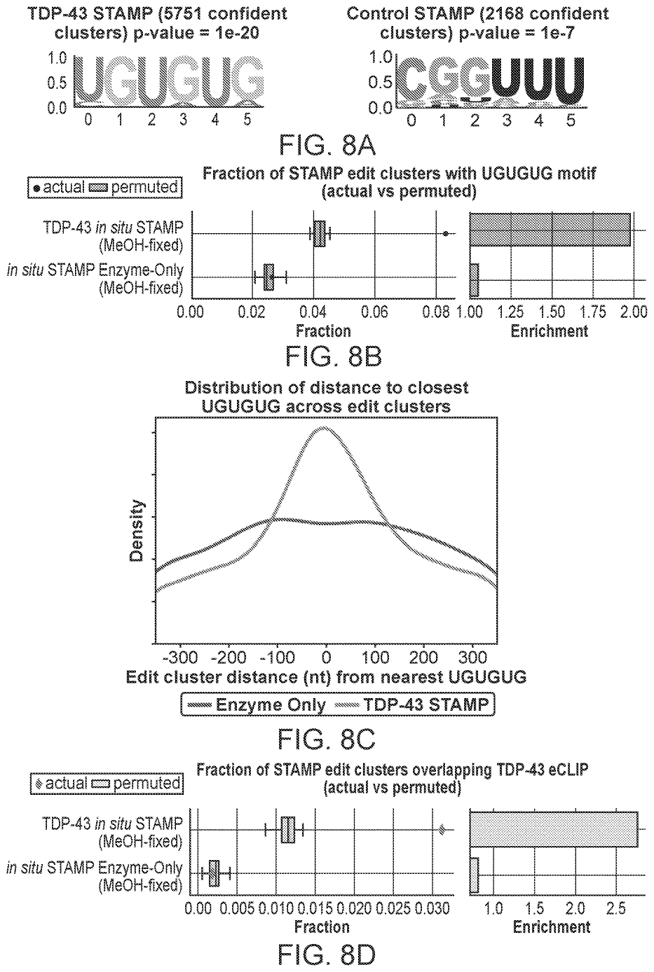

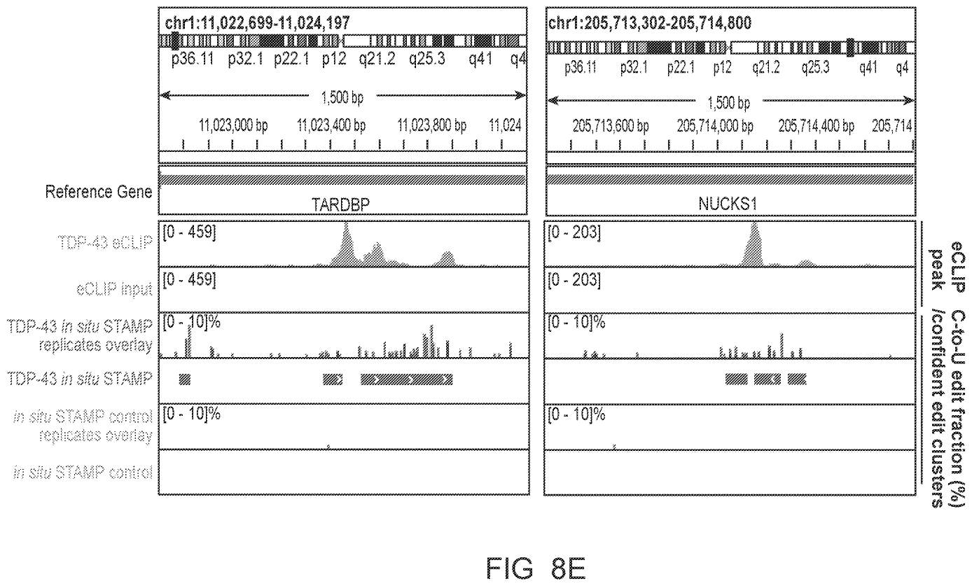

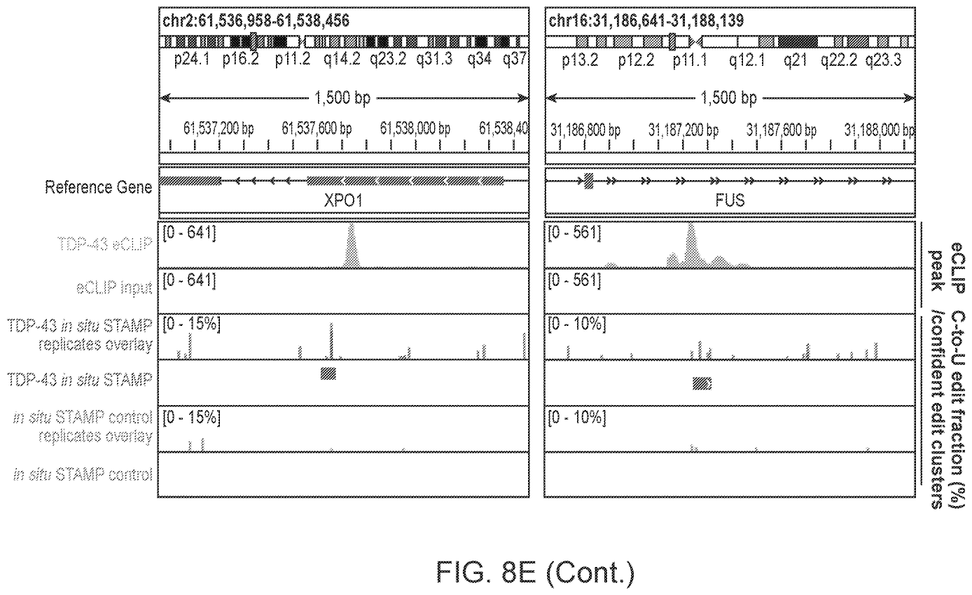

Applicant next sought to demonstrate the versatility of the APO1-Nb design and in situ STAMP's performance in profiling other RBPs in methanol-fixed HEK293T cells. Using a similar workflow as with RBFOX2, Applicant optimized the dilution factor for a rabbit-derived anti-TDP-43 antibody and successfully profiled TDP-43, a nuclear RNA/DNA-binding protein implicated in many neurodegenerative diseases. 5751 confident edit clusters were identified through FLARE, and de novo motif discovery on confident edit clusters of TDP-43 STAMP showed enrichment of the canonical TDP-43 binding motif UGUGUG (p=1e-20) (FIG. 8A). The fraction of confident TDP-43-STAMP edit clusters exhibiting a UGUGUG motif is notably greater than in shuffled size-matched peaks (FIG. 8B). The motif was also enriched in proximity of TDP-43 in situ STAMP edit clusters relative to enzyme-only controls (FIG. 8C). Moreover, Applicant noted a more than 2.5-fold increase in the proportion of TDP-43 in situ STAMP peaks overlapping with TDP-43 eCLIP, compared to enzyme-only controls (FIG. 8D). Known TDP-43 binding sites on the TARDP, NUCKS1, XPO1 and FUS genes were clearly captured by in situ STAMP C-to-U edit clusters, and showed agreement with eCLIP peaks (FIG. 8E). In conclusion, in situ STAMP demonstrated robust RBP binding target identification through confident edit clusters.

### Example 6. In Situ STAMP is Compatible with Multiple Cell Fixation Methods

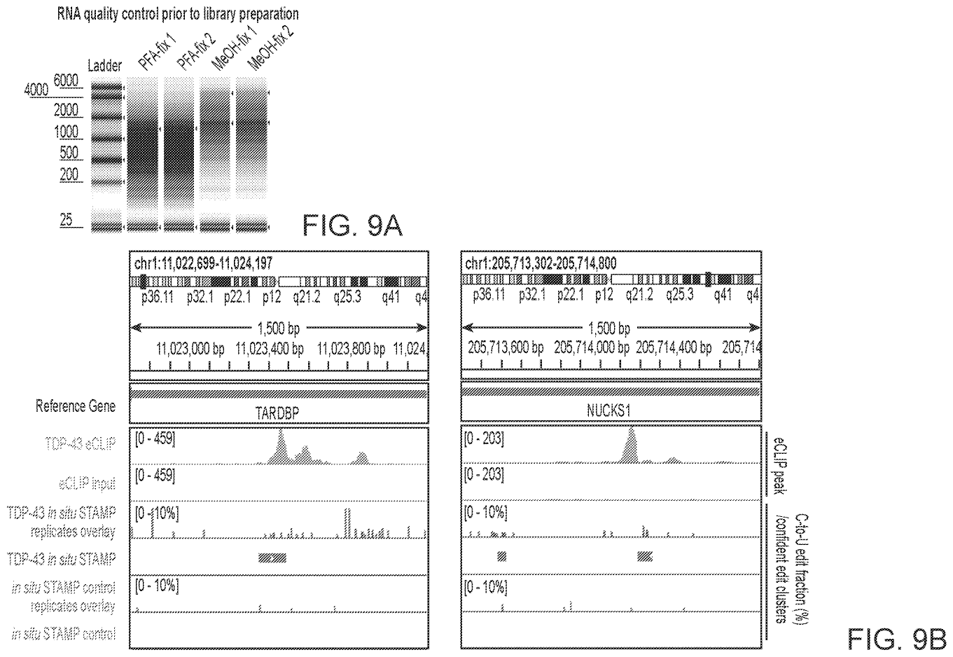

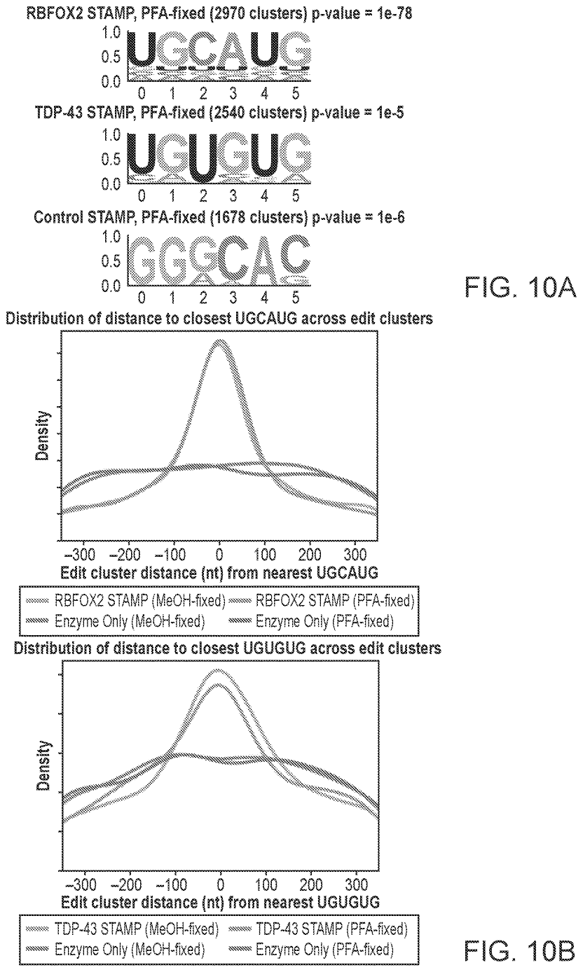

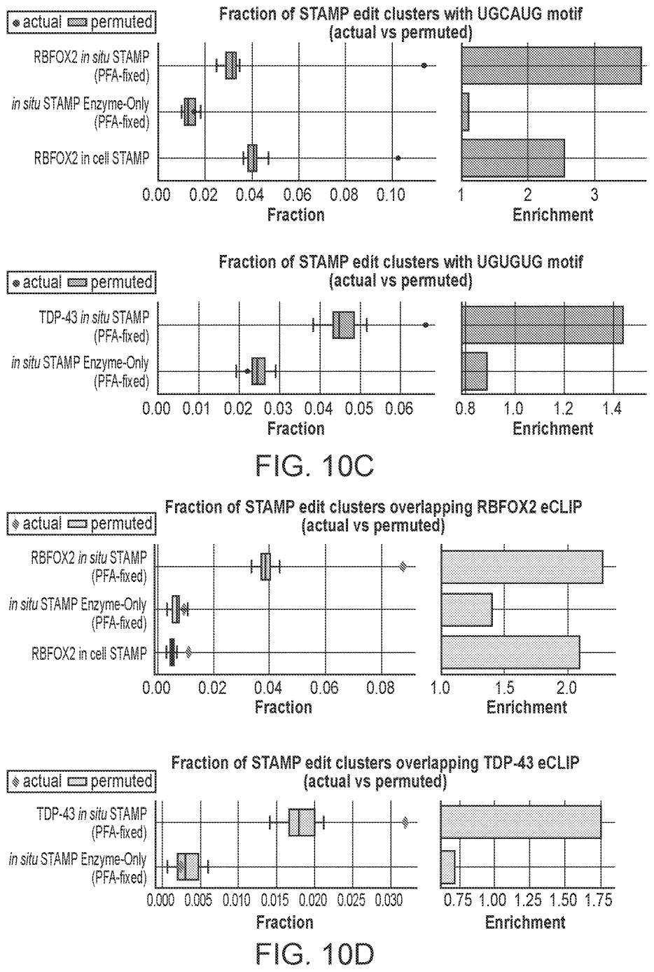

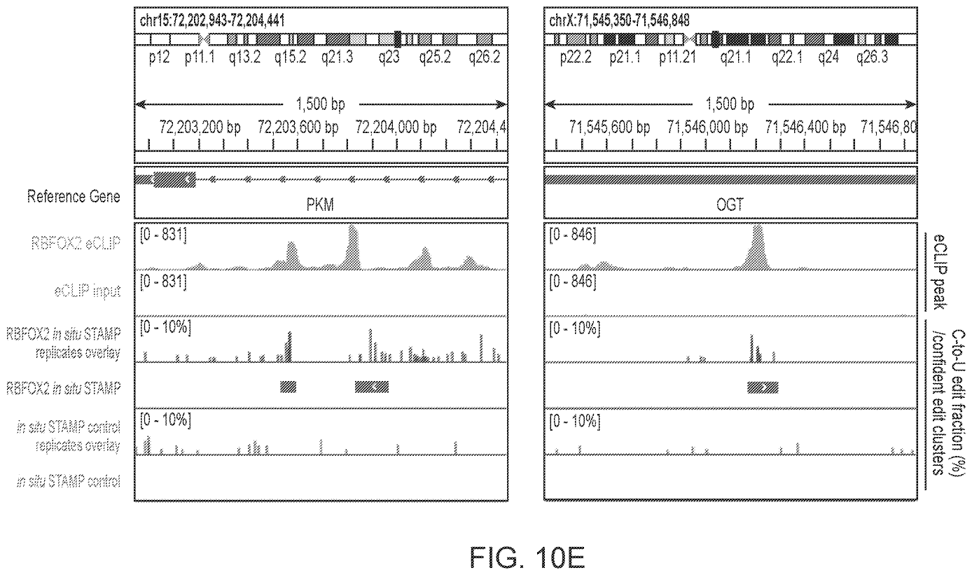

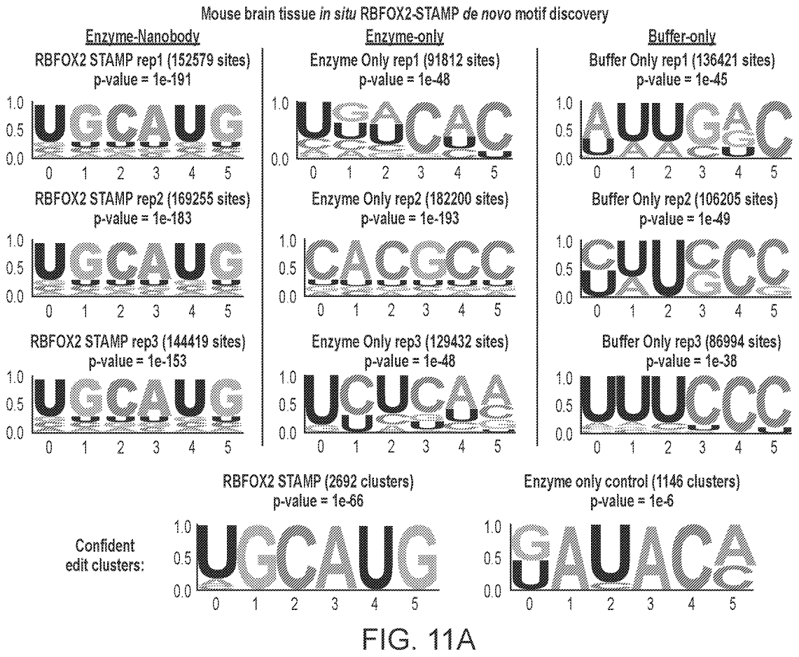

As formalin or formaldehyde fixation is more prevalent than methanol fixation in clinical sample collection workflows, Applicant next aimed to implement in situ STAMP on formaldehyde-fixed samples. Since formaldehyde fixation is known to damage RNAs and induce RNA modifications including nonspecific cytosine deamination, Applicant anticipated higher noise levels for in situ STAMP with formaldehyde fixation relative to methanol fixation. Applicant optimized a protocol to fix cells with 4% paraformaldehyde (PFA) that effectively preserved RNA integrity as judged by electrophoresis analysis with a TapeStation system (FIG. 9A). After performing in situ STAMP on PFA-fixed cells, Applicant observed comparable numbers of confident edit clusters as in methanol-fixed cells for both RBFOX2 (2970 clusters) and TDP-43 (2540 clusters). De novo motif discovery could identify both the canonical RBFOX2 motif (p=1e-78) and TDP-43 motif (p=1e-5; 2^{nd} top motif) and these motifs were enriched in regions adjacent to confident edit cluster centers comparable to methanol-fixed in situ STAMP (FIG. 10A, FIG. 10B). A similar fraction of confident edit clusters exhibited the canonical motif of each RBP in PFA-fixed samples was observed as in MeOH-fixed STAMP, although the enrichment of actual fraction versus permuted fraction was slightly lower (FIG. 10C). The confident edit clusters that overlapped with eCLIP IDR peaks in PFA-fixed HEK293T in situ STAMP displayed a similar enrichment to the observation in the MeOH-fixed HEK293T STAMP (FIG. 10D). The C-to-U editing and the confident edit clusters at representative target genes also uncovered their striking proximity and alignment with eCLIP peaks (FIG. 10E, FIG. 9B). In summary, in situ STAMP is compatible with PFA-fixed cells, enhancing its utility in analysis of clinical samples and extending its applicability to a broader range of experimental workflows.

### Example 7. In Situ STAMP Identifies RBP-RNA Interaction Sites in Fixed Tissue Samples

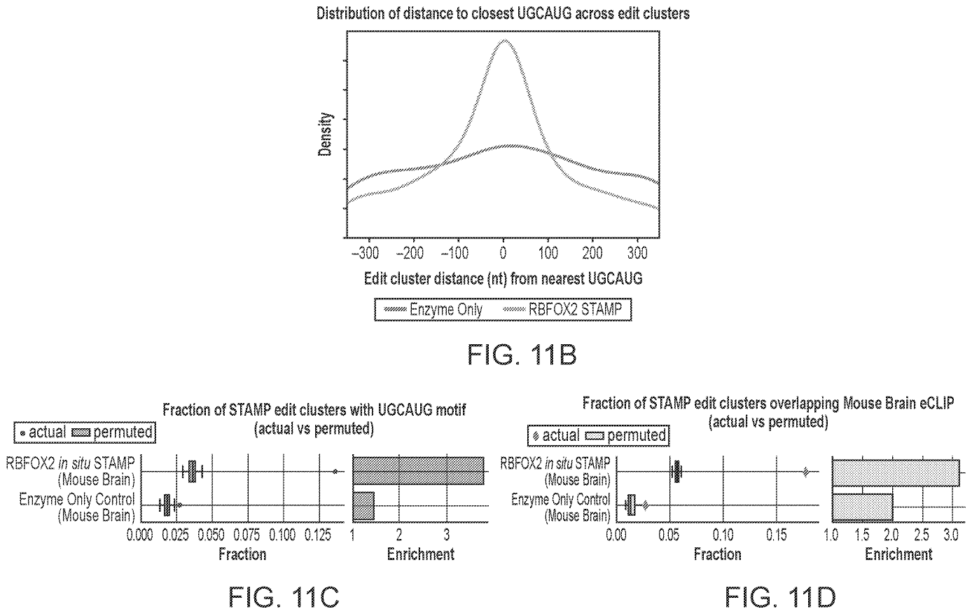

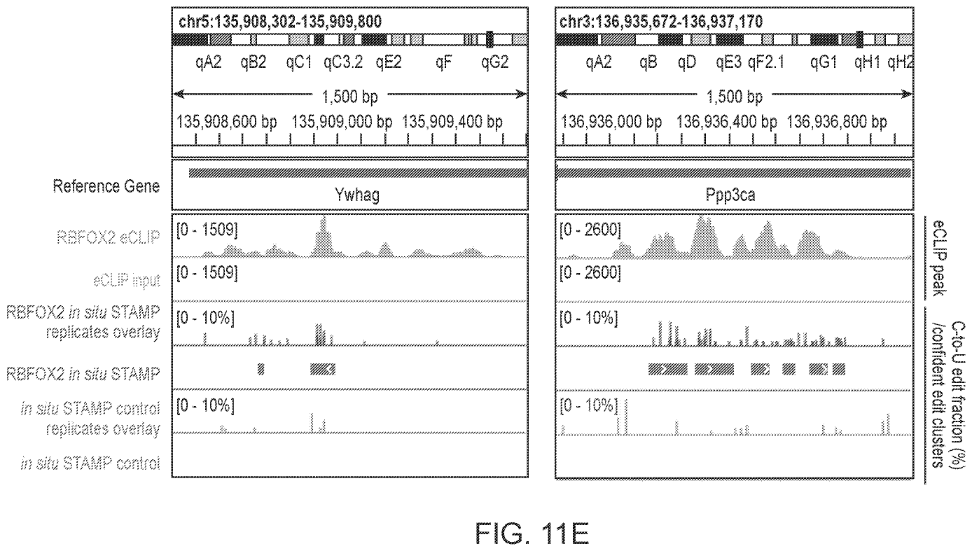

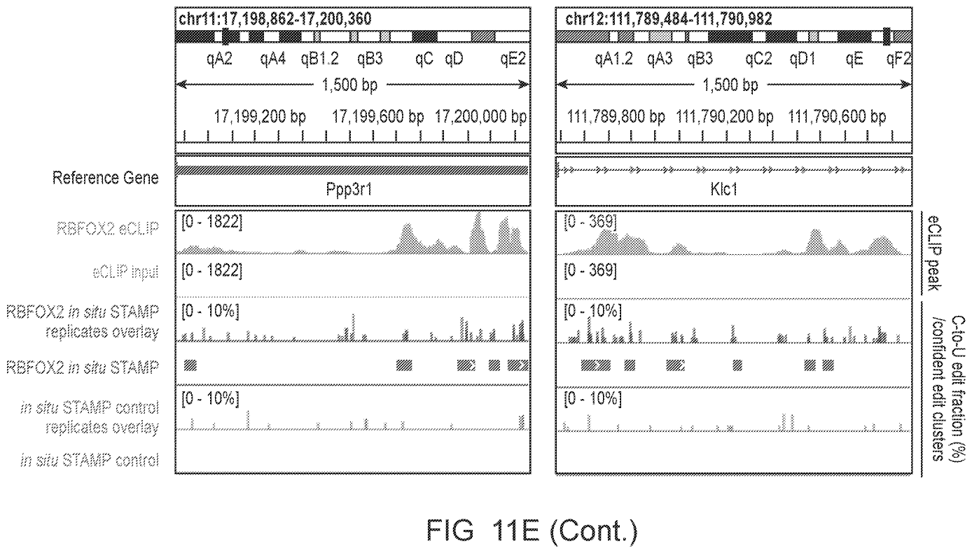

Finally, Applicant sought to determine whether in situ STAMP could capture authentic RBP-RNA interactions in the context of intact primary tissue samples. The complex nature of primary tissues may pose a challenge for in situ STAMP, as elements like the extracellular matrix might hinder binding of a primary antibody to its target RBP, recognition of the primary antibody by Apo1-Nb, or the editing reaction itself. To test in situ STAMP's compatibility with primary tissues, Applicant conducted in situ RBFOX2-STAMP in methanol-fixed 20 μm-thick mouse brain slices. Excitingly, in situ STAMP in mouse brain tissue exhibited striking performance. The canonical RBFOX2 motif (U)GCAUG was found in all replicates of RBFOX2 in situ STAMP as well as in confident edit clusters through de novo motif discovery (FIG. 11A). The RBFOX2 motif was also enriched across all confident edit clusters compared to enzyme-only controls, with around 13% of edit clusters bearing a UGCAUG motif (FIG. 11B, 11C). Furthermore, approximately 17% of confident edit clusters overlapped RBFOX2 eCLIP peaks in mouse whole brain tissue, with a >3-fold enrichment compared to permuted clusters (FIG. 11D). Additionally, upon closer examination at individual RBFOX2 target genes, a notable correlation was observed between in situ STAMP confident edit clusters and eCLIP peaks, indicating a strong alignment between the two (FIG. 11E). In conclusion, in situ STAMP can capture RBP-RNA interactions in primary tissues, potentially enabling application to a broad range of precious clinical samples.

### EQUIVALENTS

Unless otherwise defined, all technical and scientific terms used herein have the same meaning as commonly understood by one of ordinary skill in the art to which this technology belongs.

The present technology illustratively described herein may suitably be practiced in the absence of any element or elements, limitation or limitations, not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the present technology claimed.

Thus, it should be understood that the materials, methods, and examples provided here are representative of preferred aspects, are exemplary, and are not intended as limitations on the scope of the present technology.

The present technology has been described broadly and generically herein. Each of the narrower species and sub-generic groupings falling within the generic disclosure also form part of the present technology. This includes the generic description of the present technology with a proviso or negative limitation removing any subject matter from the genus, regardless of whether or not the excised material is specifically recited herein.

In addition, where features or aspects of the present technology are described in terms of Markush groups, those skilled in the art will recognize that the present technology is also thereby described in terms of any individual member or subgroup of members of the Markush group.

All publications, patent applications, patents, and other references mentioned herein are expressly incorporated by reference in their entirety, to the same extent as if each were incorporated by reference individually. In case of conflict, the present specification, including definitions, will control.

1. An engineered, non-naturally occurring recognition-editing system comprising a fusion polypeptide comprising:
(a) a protein-specific recognition module, wherein the module specifically recognizes a target protein; and
(b) a nucleic acid base editor domain,
wherein the nucleic acid base editor domain is capable of (i) altering one or more specific bases of a nucleic acid to a different base; or (ii) modifying a specific nucleoside of a nucleic acid in a manner that is detectable by nucleic acid sequencing.

2. The engineered, non-naturally occurring recognition-editing system of claim 1, further comprising a supplementary recognition component, optionally wherein the supplementary recognition component is a primary antibody that specifically binds to the target protein.

3. (canceled)

4. The engineered, non-naturally occurring recognition-editing system of claim 1, wherein the protein-specific recognition module is an antibody or an antigen binding fragment thereof, or a nanobody or an antigen binding fragment thereof, wherein the protein-specific recognition module specifically binds to the target protein, or wherein the protein-specific recognition module specifically binds to the supplementary recognition component or wherein the protein-specific recognition module is a small molecule.

5.-6. (canceled)

7. The engineered, non-naturally occurring recognition-editing system of claim 1, wherein the protein-specific recognition module is capable of recognizing a tag fused to the target protein or wherein the nucleic acid base editor domain comprises an APOBEC family base editor or an ADAR family base editor or wherein the nucleic acid base editor domain comprises an APOBEC1 enzyme.

8.-9. (canceled)

10. The engineered, non-naturally occurring recognition-editing system of claim 1, wherein the protein-specific recognition module is a nanobody, optionally wherein the nanobody specifically binds to an Immunoglobulin G (IgG), further optionally wherein the IgG is rabbit IgG.

11. (canceled)

12. (canceled)

13. The engineered, non-naturally occurring recognition-editing system of claim 1, wherein the target protein is an RNA-binding protein (RBP), optionally wherein the target protein is RBFOX2 or TDP-43.

14. (canceled)

15. The engineered, non-naturally occurring recognition-editing system of claim 1, wherein the fusion polypeptide comprises an amino acid sequence that is at least 70%, at least 75%, at least 80%, at least 85%, at least 90%, at least 95%, at least 99%, or 100% identical to SEQ ID NO: 1.

16. A method of identifying one or more nucleic acid binding site of a target protein or target protein complex in a biological sample, comprising:
(a) processing the biological sample, wherein processing comprises fixing and permeabilizing cells in the biological sample, wherein the biological sample is in a buffer;
(b) contacting the biological sample with the engineered, non-naturally occurring recognition-editing system of claim 1 for a time sufficient to permit localization of the recognition-editing system to a target protein;
(c) activating the nucleic acid base editor domain for a time sufficient to permit base editing of adjacent nucleic acids, thereby producing base edits;
(d) extracting nucleic acids from the biological sample;
(e) sequencing the nucleic acids in the biological samples, thereby generating first sequence data; and
(f) profiling the base edits by identifying one or more sequence differences in the first sequence data relative to second sequence data obtained from a biological sample that was not contacted with the engineered, non-naturally occurring recognition-editing system,
wherein the position of the one or more sequence differences represents one or more nucleic acid binding site of the target protein or the target protein complex.

17. (canceled)

18. The method of claim 16, wherein processing further comprises semi-permanently linking the target protein to its nucleic acid binding sites or wherein activating the nucleic acid base editor domain comprises converting conditions of the buffer to promote enzymatic activity of the nucleic acid base editor domain.

19.-21. (canceled)

22. The method of claim 16, wherein contacting the biological sample with the engineered, non-naturally occurring recognition-editing system comprises:
(i) contacting the biological sample with a supplementary recognition component for a time sufficient to permit the binding of the supplementary recognition component to the target protein; and
(ii) contacting the biological sample with the fusion polypeptide for a time sufficient to permit the binding of the protein-specific recognition module to the supplementary recognition component.

23. The method of claim 22, wherein contacting the biological sample with a supplementary recognition component is performed for about 1 hour.

24. The method of claim 16, wherein contacting the biological sample with the fusion polypeptide is performed for about 1 hour or wherein activating the nucleic acid base editor domain is performed for about 16 hours.

25. (canceled)

26. A fusion polypeptide comprising:
(a) a protein-specific recognition module; and
(b) a nucleic acid base editor domain,
wherein the nucleic acid base editor domain is capable of (i) altering one or more specific bases of a nucleic acid to a different base; or (ii) modifying a specific nucleoside of a nucleic acid in a manner that is detectable by nucleic acid sequencing.

27. The fusion polypeptide of claim 26, wherein the protein-specific recognition module is an antibody or an antigen binding fragment thereof, or a nanobody or an antigen binding fragment thereof, wherein the protein-specific recognition module specifically binds to a target protein.

28. The fusion polypeptide of claim 26, wherein the protein-specific recognition module is an antibody or an antigen binding fragment thereof, or a nanobody or an antigen binding fragment thereof, wherein the protein-specific recognition module specifically binds to an Immunoglobulin G (IgG) or wherein the protein-specific recognition module is a nanobody, optionally wherein the nanobody specifically binds to an Immunoglobulin G (IgG).

29.-30. (canceled)

31. The fusion polypeptide of claim 26, wherein the nucleic acid base editor domain comprises an APOBEC family base editor or an ADAR family base editor or wherein the nucleic acid base editor domain comprises an APOBEC1 enzyme or wherein the fusion polypeptide comprises an amino acid sequence that is at least 70%, at least 75%, at least 80%, at least 85%, at least 90%, at least 95%, at least 99%, or 100% identical to SEQ ID NO: 1.

32.-33. (canceled)

34. A polynucleotide encoding the fusion polypeptide of claim 26.

35. A vector or cell comprising the polynucleotide of claim 34.

36.-38. (canceled)

39. A kit comprising one or more of the recognition-editing system of any one of claim 1.
